(19) United States
(12) Patent Application Publication
(10) Pub. No.: US 2009/0238768 A1
(43) Pub. Date: Sep. 24, 2009

(54) MULTIMERIC MAGNETIC RESONANCE CONTRAST AGENTS

(76) Inventors: Oskar Axelsson, Hagan (NO); Haukur Johannesson, Oslo (NO); Andreas Meijer, Oslo (NO); Andreas Olsson, Oslo (NO); Tim Stahlberg, Sodertalje (SE); Mikkel Thaning, Oslo (NO); Duncan Wynn, Buckinghamshire (GB); Anders Brathe, Oslo (NO)

Correspondence Address: GE HEALTHCARE, INC., IP DEPARTMENT 101 CARNEGIE CENTER, PRINCETON, NJ, 08540-6231, US

(21) Appl. No.: 12/095,218

(22) PCT Filed: Dec. 1, 2006
(86) PCT No.: PCT/NO06/00450

§ 371 Date: May 28, 2008

(30) Foreign Application Priority Data
Dec. 2, 2005 (NO) 20055703
Dec. 2, 2005 (NO) 20055704
Sep. 21, 2006 (NO) 20064269
Oct. 6, 2006 (NO) 20064539

(51) Int. Cl. A61K 49/10; C07F 5/00

(52) U.S. Cl. 424/9.361; 534/15; 424/9.36

(57) ABSTRACT

The present invention relates to novel compounds of formula (I) and (II), compositions comprising compounds of formula (II) and their use as contrast agents in magnetic resonance (MR) imaging (MRI) and MR spectroscopy (MRS).

The present invention relates to novel compounds of formula (I) and (II), compositions comprising compounds of formula (II) and their use as contrast agents in magnetic resonance (MR) imaging (MRI) and MR spectroscopy (MRS).

MR image signal is influenced by a number of parameters that can be divided into two general categories: inherent tissue parameters and user-selectable imaging parameters. Inherent tissue parameters that affect MR signal intensity of a particular tissue are mainly the proton density, i.e. hydrogen nuclei density of that tissue and its inherent T₁ and T₂ relaxation times. Signal intensity is also influenced by other factors such as flow. The contrast between two adjacent tissues, e.g. a tumour and normal tissue depends on the difference in signal between the two tissues. This difference can be maximised by proper use of user-selectable parameters. User-selectable parameters that can affect MR image contrast include choice of pulse sequences, flip angles, echo time, repetition time and use of contrast agents.

Contrast agents are often used in MRI in order to improve the image contrast. Contrast agents work by effecting the T₁, T₂ and/or T₂* relaxation times and thereby influencing the contrast in the images. Information related to perfusion, permeability and cellular density as well as other physiological parameters can be obtained by observing the dynamic behaviour of a contrast agent.

Several types of contrast agents have been used in MRI. Water-soluble paramagnetic metal chelates, for instance gadolinium chelates like Omniscan™ (GE Healthcare) are widely used MR contrast agents. Because of their low molecular weight they rapidly distribute into the extracellular space (i.e. the blood and the interstitium) when administered into the vasculature. They are also cleared relatively rapidly from the body.

Blood pool MR contrast agents on the other hand, for instance superparamagnetic iron oxide particles, are retained within the vasculature for a prolonged time. They have proven to be extremely useful to enhance contrast in the liver but also to detect capillary permeability abnormalities, e.g. “leaky” capillary walls in tumours which are a result of tumour angiogenesis.

The existent paramagnetic metal chelates that are used as MR contrast agents have a low relaxivity at the 1.5 T magnetic field that is standard in most of today's MR scanner. In 3 T systems which probably will dominate or at least be a substantial fraction of the market in the future, the intrinsic contrast is lower, all T₁ values are higher and the hardware will be faster, so the need for a contrast agent with good performance at 3 T is considerable. In general, the longitudinal relaxivity (rl) of contrast agents falls off at the high magnetic fields of the modern MR scanners, i.e. 1.5 T, 3 T or even higher. This is due to the fast rotational Brownian motion of small molecules in solution which leads to weaker magnetic field coupling of the paramagnetic metal ion to the water molecules than anticipated.

Many attempts have been made to produce contrast agents with high relaxivity by incorporating the paramagnetic metal chelates into larger molecules, such as various polymers. These attempts have been of limited success because of fast internal rotations or segmental motions. Another approach are paramagnetic metal chelates that are bound to or do bind to proteins. However such compounds suffer from pharmacological and pharmacokinetic disadvantages like long excretion time or the risk for interactions with protein bound drugs. Further the leakage through normal endothelium into the interstitium is still substantial.

The present invention provides novel compounds that perform well as MR contrast agents at high magnetic fields, i.e. above 1.5 T. The novel compounds are trimeric rigid structures that have slowly rotating bonds and in addition show high water exchange rates.

US-A1-2004/0265236 discloses trimeric macrocyclic substituted benzene derivatives which contain bonds with free rotation. Particularly, there are one or more methylene groups in the side chains of these compounds that would allow the compounds to rotate freely. The trimeric macrocyclic substituted benzene derivatives have decreased relaxivity compared to the compounds of the present invention due to the presence of bonds with free rotation.

We have now developed contrast agents with high relaxivity for use in MR imaging and MR spectroscopy, particularly performed under high magnetic field strength, e.g. at a field strength of 1.5 T, 3 T or above.

Thus, in a first aspect the invention provides compounds of formula (I) consisting of a core and groups —R-L-X attached to said core

A-(R-L-X)n (I)

wherein
A denotes a rigid core;
R is present or not and if present is the same or different and denotes a moiety that that constitutes an obstacle for the rotation of the covalent bond between the core A and R and/or the covalent bond between R and L and/or L and X, if L is present and/or the covalent bond between R and X, if L is not present;
L is present or not and if present is the same or different and denotes a linker moiety;
X is the same or different and denotes a chelator; and
n denotes an integer of 3 or 4.

The term “chelator” denotes a chemical entity that binds (complexes) a metal ion to form a chelate. If the metal ion is a paramagnetic metal ion, the chemical entity, i.e. complex formed by said paramagnetic metal ion and said chelator is denoted a “paramagnetic chelate”.

A preferred embodiment of a compound of formula (I) is a compound of formula (II) consisting of a core and groups —R-L-X′ attached to said core

A-(R-L-X′)n (II)

wherein
A denotes a rigid core;
R is the same or different and denotes a moiety that that constitutes an obstacle for the rotation of the covalent bond between the core A and R and/or the covalent bond between R and L and/or L and X, if L is present and/or the covalent bond between R and X′, if L is not present;
L is present or not and if present is the same or different and denotes a linker moiety;
X′ is the same or different and denotes a paramagnetic chelate consisting of a chelator X and a paramagnetic metal ion M; and
n denotes an integer of 3 or 4.

In said preferred embodiment, said paramagnetic chelate X′ consists of the chelator X and a paramagnetic metal ion M, said chelator X and paramagnetic metal ion M form a complex which is denoted a paramagnetic chelate.

In the following the term “ . . . X/X′”, e.g. in R-L-X/X′ or in the formulae means that the statement made or the drawn formula is equally suitable for compounds or residues comprising the chelator X or the paramagnetic chelate X′.

Compounds of formula (I) and (II) are rigid compounds since they comprise a rigid core A. There are various known molecules in organic chemistry that may fulfil this criterion. Preferably A is a non-polymeric rigid core. In another preferred embodiment, A is a cyclic core or a carbon atom having attached thereto 3 or 4 groups R-L-X/X′, wherein, when 3 groups R-L-X/X′ are attached to said carbon atom, the forth valence may be hydrogen or a group selected from amino, hydroxyl, C₁-C₃-alkyl or halogen.

In One embodiment A is preferably a saturated or non-saturated, aromatic or aliphatic ring comprising at least 3 carbon atoms and optionally one or more heteroatoms N, S or O, said ring being optionally substituted with one or more of the following substituents: C₁-C₃-alkyl, optionally substituted with hydroxyl or amino groups, amino or hydroxyl groups or halogen, provided that there are n attachment points left for groups R-L-X/X′. Preferably, A is an aliphatic saturated or non-saturated 3- to 10-membered ring like cyclopropane, cyclobutane, cycloheptan or cyclohexane, which optionally comprises one or more heteroatoms N, S or O and which is optionally substituted with one or more substituents C₁-C₃-alkyl, optionally substituted with hydroxyl or amino groups, amino or hydroxyl groups or halogen, provided that there are 3 or 4 attachment points left for pendant groups R-L-X/X′. Alternatively, A is an aliphatic 3- to 10-membered ring optionally comprising one or more heteroatoms N, S, or O wherein one or more of the ring carbon atoms are carbonyl groups.

In another preferred embodiment, A is an aromatic single or fused 5- to 10-membered ring optionally comprising one or more heteroatoms N, S or O. Examples for such rings are for instance benzene or naphthalene. The aforementioned rings are optionally substituted with one or more substituents C₁-C₃-alkyl, optionally substituted with hydroxyl or amino groups, amino or hydroxyl groups or halogen, provided that there are at 3 or 4 attachment points left for pendant groups R-L-X/X′.

Further, compounds of formula (I) and (II) are rigid compounds since the R-L-X/X′ pendant groups of formula (I) and (II) exert a rotation restriction on the covalent bond between the core A and R and/or the covalent bond between R and L and/or L and X/X′, if L is present and/or the covalent bond between R and X/X′, if L is not present, such that these bonds rotate preferably less than 10⁷ times/second at 37° C.

In the compounds of formula (I) and (II), R is the same or different and denotes a moiety that that constitutes an obstacle for the rotation of the covalent bonds between the core A and R and/or the covalent bond between R and L and/or L and X/X′, if L is present and/or the covalent bond between R and X/X′, if L is not present. This may be achieved in different ways, e.g. by a) choosing a moiety R which is a slowly rotating moiety or b) choosing a moiety R whose rotation is hindered by sterical interaction with the core, and/or L, if present and/or X/X′ and/or other R groups.

Regarding a) the term “slowly rotating moiety” denotes a moiety with a conformational lifetime of more than 0.1 μs. Preferred slowly rotating moieties and thus preferred R are substituted aromatic amides such as N-methylanilides.

Regarding b) such sterical interaction occurs if R is a bulky moiety like an at least 5-membered carbocyclic or heterocyclic ring or a bicyclical or polycyclic ring. Such sterical interaction may further be promoted by using a bulky moiety R, e.g. the aforementioned bulky moieties which is substituted with C₁-C₃-alkyl, e.g. methyl, ethyl, n-propyl or isopropyl. Such bulky moieties R hinder the rotation of the R group due to interaction with one or more other R moieties and/or T and/or X/X′ and/or L, if present.

In a preferred embodiment R is selected from a residue of an optionally substituted aromatic or non-aromatic 5- to 7-membered carbocyclic or heterocyclic ring like pyridinyl, phenyl, substituted phenyl like benzyl, ethylbenzyl or cyclohexyl. In another preferred embodiment R is selected from a residue of an optionally substituted bicyclical or polycyclic ring like naphthyl or benzimidazolyl. Optional substituents are C₁-C₈-alkyl, hydroxyl, amino or mercapto groups or C₁-C₈-alkyl containing one or more hydroxyl or amino groups like CH₂OH, C₂H₄OH, CH₂NH₂ and/or an oxo-group like CH₂OCH₃ or OC₂H₄OH.

The term “residues of . . . ” in the previous paragraph is chosen since R is attached to A and L, if L is present or X/X′, if L is not present. Thus, R is to be seen as a residue.

In a particularly preferred embodiment R is a residue of a substituted 6-membered aromatic ring, preferably a residue of a 6-membered aromatic ring comprising a methyl or ethyl group.

R is attached to the core A either via a covalent bond or via covalent bonds. The former denotes a single covalent bond while the latter denotes a situation where R is attached to the core A by more than one single covalent bond. This is the case when R is a cyclic moiety which is has two attachment points at the core A, i.e. which is fused to the core A. This is exemplified in formula IIIa wherein A is a phenyl core having attached thereto 3 R (in bold font) in the form of fused rings:

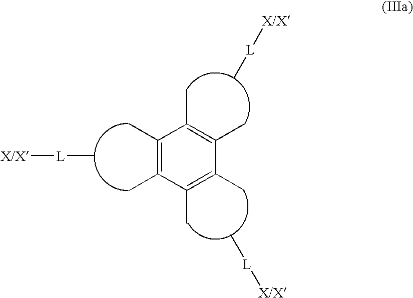

Alternatively, R is attached to A via the moiety of formula (IIIb)

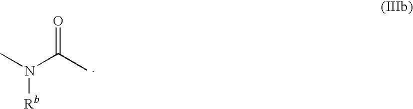

wherein
R^{b} stands for H, C₁-C₈-alkyl, optionally substituted with one or more hydroxyl or amino groups.

Preferably, R^{b} stands for H, C₁-C₃-alkyl, e.g. methyl, ethyl, n-propyl or isopropyl, optionally substituted with one or more hydroxyl or amino groups, e.g. CH₂OH, C₂H₄OH, CH₂NH₂ or C₂H₄NH₂.

In formula (IIIb), either the nitrogen atom or the carbonyl group may be attached to the core A. Preferably, the carbonyl group is attached to the core A.

In another preferred embodiment all R are the same.

In compounds of formula (I) and (II), L may be present or not. If L is not present, R is directly linked to X (compounds of formula (I)) or X′ (compounds of formula (II)) via a covalent bond. If L is present, each L is the same or different and denotes a linker moiety, i.e. a moiety that is able to link A and X/X′ and R and X/X′, respectively.

Preferred examples of L are:

Linker moieties —(CZ¹Z²)ₘ—

wherein
m is an integer of 1 to 6; and
Z¹ and Z² independently of each other denote a hydrogen atom, a hydroxyl group or a C₁-C₈-allyl group optionally substituted by hydroxyl, amino or mercapto groups, e.g. CH₂OH and CH₂CH₂NH₂ and/or optionally comprising an oxo-group, e.g. CH₂OCH₃ and OCH₂CH₂OH.

Linker moieties —CZ¹Z²-CO—N(R^{b})—* which are more preferred linker moieties,

wherein
* denotes the attachment of R to said linker moiety; and
Z¹, Z² and R^{b} have the meaning mentioned above

In a preferred embodiment, Z¹ and Z² are hydrogen or Z¹ is hydrogen and Z² is methyl and R^{b} is H, C₁-C₃-alkyl, e.g. methyl, ethyl, n-propyl or isopropyl, optionally substituted with one or more hydroxyl or amino groups, e.g. CH₂OH, C₂H₄OH, CH₂NH₂ or C₂H₄NH₂.

Linker moieties *—CO—N(R^{b})—*

wherein
denotes the attachment of R to said linker moiety, i.e. R is either attached to the carbon atom or the nitrogen atom of said linker moiety; and
R^{b} has the meaning as above.

Linker moieties —CO—CZ¹Z²-N(R^{b})—*

wherein
* denotes the attachment of R to said linker moiety; and
Z¹, Z² and R^{b} have the meaning mentioned above

In a preferred embodiment, Z¹ and Z² are hydrogen or Z¹ is hydrogen and Z² is methyl and R^{b} is H, C₁-C₃-alkyl, e.g. methyl, ethyl, n-propyl or isopropyl, optionally substituted with one or more hydroxyl or amino groups, e.g. CH₂OH, C₂H₄OH, CH₂NH₂ or C₂H₄NH₂.

Linker moieties which are amino acid residues —CH₂—CO—NH—CH(Z¹)CO—NH—* wherein

* denotes the attachment of R to said linker moiety; and
Z³ stands for the side group of the naturally occurring α-amino acids.

Further preferred examples of L are or comprise residues of benzene or N-heterocycles such as imidazoles, triazoles, pyrazinones, pyrimidines and piperidines, wherein R is attached to one of the nitrogen atoms in said N-heterocycles or to a carbon atom in said N-heterocycles or in benzene.

If L comprises one of the aforementioned residues, i.e. benzene or an N-heterocycle, L is preferably

—*N-heterocycle-(CZ¹Z²)ₘ- or -*benzene-(CZ¹Z²)ₘ—

wherein
R is attached to one of the nitrogen atoms in said N-heterocycle or to a carbon atom in said benzene; and
Z¹, Z² and m are as defined above.

Preferred examples of such linker moieties L are:

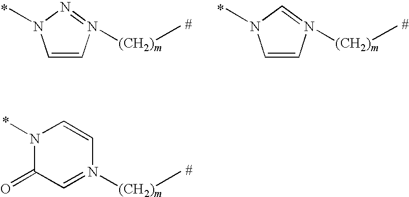

wherein * denotes the attachment of R to said linker moiety, # denotes the attachment of X/X′ to said linker moiety and m is 1 or 2.

Preferably, if present, all L are the same.

In compounds of formula (I), X is the same or different and denotes a chelator. In the preferred embodiment of compounds of formula (II), X is X′ which stands for a paramagnetic chelate, i.e. a chelator X which forms a complex with a paramagnetic metal ion M. Numerous chelators X which form complexes with paramagnetic metal ions M are known in the art. Preferably, X is a cyclic chelator of formula (IV):

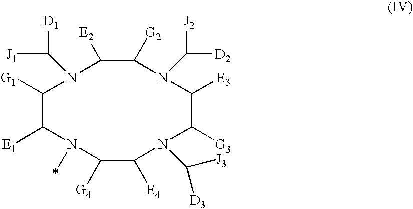

wherein
* denotes the attachment of L, if present, or the core, if L is not present;
E₁ to E₄ independent of each other is selected from H, CH₂, CH₃, OCH₃, CH₂OH, CH₂OCH₃, OCH₂CH₃, OCH₂CH₂OH, COOH, COOCH₃, COOCH₂CH₃, C(O)NH₂, C(O)N(CH₃)₂, C(O)N(CH₂CH₃)CH₃ or C(O)N(CH₂CH₃)₂;
G₁ to G₄ independent of each other is selected from H, CH₂, CH₃, OCH₃, CH₂OH, CH₂OCH₃, OCH₂CH₃, OCH₂CH₂OH, COOH, COOCH₃, COOCH₂CH₃, C(O)NH₂, C(O)N(CH₃)₂, C(O)N(CH₂CH₃)CH₃, or C(O)N(CH₂CH₃)₂;
D₁ to D₃ independent of each other is selected from H, OH, CH₃, CH₂CH₃, CH₂OH, CH₂OCH₃, OCH₂CH₃, OCH₂CH₂OH or OCH₂C₆H₅; and
J₁ to J₃ independent of each other is selected from COOH, P(O)(OH)₂, P(O)(OH)CH₃, P(O)(OH)CH₂CH₃, P(O)(OH)(CH₂)₃CH₃, P(O)(OH)Ph, P(O)(OH)CH₂Ph, P(O)(OH)OCH₂CH₃, CH(OH)CH₃, CH(OH)CH₂OH, C(O)NH₂, C(O)NHCH₃, C(O)NH(CH₂)₂CH₃, OH or H.

Preferred chelators X are residues of diethylenetriaminopentaacetic acid (DTPA), N-[2-[bis(carboxymethyl)amino]-3-(4-ethoxyphenyl)propyl]-N-[2-[bis(carboxymethyl)-amino]ethyl]-L-glycine (EOB-DTPA), N,N-bis[2-[bis(carboxymethyl)amino]-ethyl]-L-glutamic acid (DTPA-Glu), N,N-bis[2-[bis(carboxymethyl)amino]-ethyl]-L-lysine (DTPA-Lys), mono- or bis-amide derivatives of DTPA such as N,N-bis[2-[carboxymethyl[(methylcarbamoyl)methyl]amino]-ethyl]glycine (DTPA-BMA), 4-carboxy-5,8,11-tris(carboxymethyl)-1-phenyl-2oxa-5,8,11-triazamidecan-13-oic acid (BOPTA), DTPA BOPTA, 1,4,7,10-tetraazacyclododecan-1,4,7-triactetic acid (DO3A), 1,4,7,10-tetraazacyclododecan-1,4,7,10-tetraactetic acid (DOTA), ethylenediaminotetraacetic acid (EDTA), 10-(2-hydroxypropyl)-1,4,7,10-tetraazacyclododecan-1,4,7-triacetic acid (HPDO3A), 2-methyl-1,4,7,10-tetraazacyclododecan-1,4,7,10-tetraacetic acid (MCTA), tetramethyl-1,4,7,10-tetraazacyclododecan-1,4,7,10-tetraacetic acid (DOTMA), 3,6,9,15-tetraazabicyclo[9.3.1]pentadeca-1(15), 11,13-triene-3,6,9-triacetic acid (PCTA), PCTA 12, cyclo-PCTA 12, N,N′Bis(2-aminoethyl)-1,2-ethanediamine (TETA), 1,4,7,10-tetraazacyclotridecane-N,N′,N″,N′″-tetraacetic acid (TRITA), 1,12-dicarbonyl, 15-(4-isothiocyanatobenzyl) 1,4,7,10,13-pentaazacyclohexadecane-N,N′, N″triaceticacid (HETA), 1,4,7,10-tetraazacyclododecane-N,N′,N″,N′″-tetraacetic acid mono-(N-hydroxysuccinimidyl) ester (DOTA-NHS), N,N′-Bis(2-aminoethyl)-1,2-ethanediamine-N-hydroxy-succinimide ester (TETA-NHS), [(2S,5S,8S,11S)-4,7,10-tris-carboxymethyl-2,5,8,11-tetramethyl-1,4,7,10-tetraazacyclododecan-1-yl]acetic acid (M4DOTA), [(2S,5S,8S,11S)-4,7-bis-carboxymethyl-2,5,8,11-tetramethyl-1,4,7,10-tetraazacyclo-dodecan-1-yl]acetic acid, (M4DO3A), (R)-2-[(2S,5S,8S,11S)-4,7,10-tris-((R)-1-carboxyethyl)-2,5,8,11-tetramethyl-1,4,7,10-tetraazacyclododecan-1-yl]propionic acid (M4DOTMA), 10-Phosphonomethyl-1,4,7,1-O-tetraazacyclododecane-1,4,7-triacetic acid (MPDO3A), hydroxybenzyl-ethylenediamine-diacetic acid (HBED) and N,N′-ethylenebis-[2-(o-hydroxyphenolic)glycine] (EHPG).

The term “residues of . . . ” in the previous paragraph is chosen since the chelator is attached to the remainder of the molecule that represents compounds of formula (I) and (II), thus X is to be seen as a residue. The attachment point of X to said remainder of the molecule that represents compounds of formula (I) and (II) may be any suitable point, e.g. a functional group like a COOH group in a chelator like DTPA, EDTA or DOTA or an amino group in a chelators like DTPA-Lys, but also a non-functional group like a methylene group in a chelators like DOTA.

Suitable chelators X and their synthesis are described in e.g. EP-A-071564, EP-A-448191, WO-A-02/48119, U.S. Pat. No. 6,399,043, WO-A-01/51095, EP-A-203962, EP-A-292689, EP-A-425571, EP-A-230893, EP-A-405704, EP-A-290047, U.S. Pat. No. 6,123,920, US-A-2002/0090342, U.S. Pat. No. 6,403,055, WO-A-02/40060, U.S. Pat. No. 6,458,337, U.S. Pat. No. 6,264,914, U.S. Pat. No. 6,221,334, WO-A-95/31444, U.S. Pat. No. 5,573,752, U.S. Pat. No. 5,358,704 and US-A-2002/0127181, the content of which are incorporated herein by reference.

In a more preferred embodiment of the present invention X is selected from residues of DOTA, DTPA, BOPTA, DO3A, HPDO3A, MCTA, DOTMA, DTPA BMA, M4DOTA, M4DO3A, PCTA, TETA, TRITA, HETA, DPDP, EDTA or EDTP.

In a particularly preferred embodiment X is selected from residues of DTPA, DOTA, BOPTA, DO3A, HPDO3A, DOTMA, PCTA, DTPA BMA, M4DOTA or M4DO3A.

As stated above, in a preferred embodiment of X, i.e. X′, the chelator X forms a complex, i.e. paramagnetic chelate, with a paramagnetic metal ion M. Suitably, M is a paramagnetic ion of a transition metal or a lanthanide metal, i.e. metals of atomic numbers 21 to 29, 42, 43, 44 or 57 to 71. More preferred, M is a paramagnetic ion of Mn, Fe, Co, Ni, Eu, Gd, Dy, Tm and Yb, particularly preferred a paramagnetic ion of Mn, Fe, Eu, Gd and Dy. Most preferably M is selected from Gd³⁺, Mn²⁺, Fe³⁺, Dy³⁺ and Eu³⁺ with Gd³⁺ being the most preferred paramagnetic ion M.

When modelling or mimicking the behaviour of compounds of formula (I) or (II) with theoretical methods and computational techniques (molecular modelling), in a preferred embodiment these compounds can be inscribed in a sphere with a diameter of from 2 to 3.5 nm and preferably in a sphere with a diameter of from 2 to 2.5 nm when using a molecular modelling software that is based on MM3 force field theoretical methods (e.g. the Spartan software) and the compounds are modelled in vacuum.

Preferred compounds of formula (I) and (II) wherein n is 3 are compounds of formula (V) and (VI), consisting of a cyanuric acid core and groups —R-L-X attached to said core

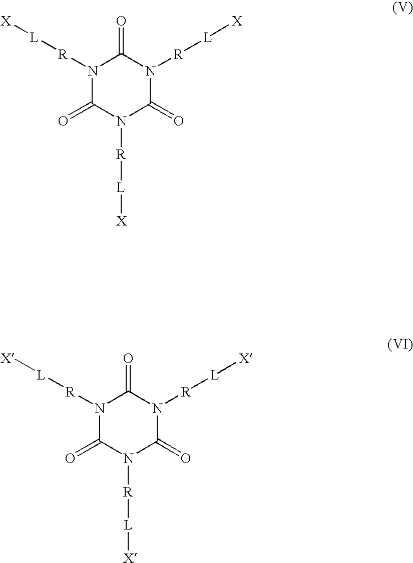

wherein R, L, X and X′ are as defined above and all R, L, X and X′ are the same.

In a preferred embodiment of compounds of formula (V) and (VI), R is a residue of an optionally substituted aromatic or non-aromatic 5- to 7-membered carbocyclic or heterocyclic ring like pyridinyl, phenyl, substituted phenyl like benzyl, ethylbenzyl or cyclohexyl. In another preferred embodiment R is selected from a residue of an optionally substituted bicyclical or polycyclic ring like naphthyl or benzimidazolyl. Optional substituents are C₁-C₈-alkyl, hydroxyl, amino or mercapto groups or C₁-C₈-alkyl containing one or more hydroxyl or amino groups like CH₂OH, C₂H₄OH, CH₂NH₂ and/or an oxo-group like CH₂OCH₃ or OC₂H₄OH.

In a more preferred embodiment, R is a residue of a substituted 6-membered aromatic ring, e.g. benzyl or preferably a residue of a 6-membered aromatic ring comprising a methyl or ethyl group like benzyl or ethylphenyl.

In a preferred embodiment of compounds of formula (V) and (VI), L is a linker moiety

—CZ¹Z²-CO—N(R^{b})—*

wherein
* denotes the attachment of R to said linker moiety; and
Z¹, Z² and R^{b} have the meaning mentioned above.

In a preferred embodiment, Z¹ and Z² are hydrogen or Z¹ is hydrogen and Z² is methyl and R¹ is H, C₁-C₃-alkyl, e.g. methyl, ethyl, n-propyl or isopropyl, optionally substituted with one or more hydroxyl or amino groups, e.g. CH₂OH, C₂H₄OH, CH₂NH₂ or C₂H₄NH₂

In another preferred embodiment, L is a residue of a N-heterocycles such as imidazole, triazole, pyrazinone, pyrimidine and piperidine, wherein R is attached to one of the nitrogen atoms in said N-heterocycle.

In another preferred embodiment, L is one of the following linker moieties:

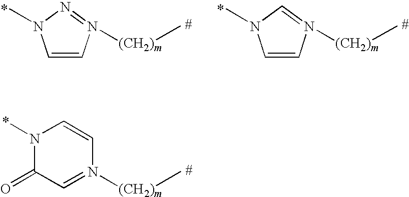

wherein * denotes the attachment of R to said linker moiety, # denotes the attachment of X/X′ to said linker moiety and m is 1 or 2.

In a preferred embodiment of compounds of formula (V) and (VI), X is selected from residues of DOTA, DTPA, BOPTA, DO3A, HPDO3A, MCTA, DOTMA, DTPA BMA, M4DOTA, M4DO3A, PCTA, TETA, TRITA, HETA, DPDP, EDTA or EDTP.

More preferably, X is selected from residues of DTPA, DOTA, BOPTA, DO3A, HPDO3A, DOTMA, PCTA, DTPA BMA, M4DOTA or M4DO3A. In a most preferred embodiment, X is a chelator of formula (IV).

In a preferred embodiment of compounds of formula (VI), M is selected from Gd³⁺, Mn²⁺, Fe³⁺, Dy³⁺ and Eu³⁺ with Gd³⁺ being the most preferred paramagnetic ion M.

In a preferred embodiment of compounds of formula (V) and (VI) all R are the same, all L are the same, all X are the same and all X′ are the same.

Further preferred compounds of formula (I) and (II) wherein n is 3 are compounds of formula (VII) and (VIII), consisting of a phenyl core (substituted, if T is not hydrogen) and groups —R-L-X attached to said core

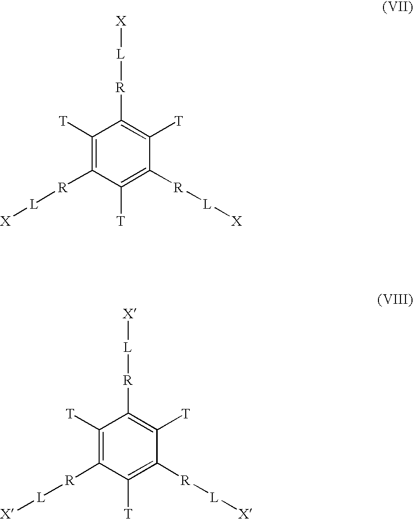

wherein
R, L, X and X′ are as defined above; and
T is the same or different and denotes a single atom or small group.

If T is a small group, it is preferably a small organic group having a molecular weight of less than 100 Da. In a more preferred embodiment, T is selected from C₁-C₃-alkyl, e.g. methyl, ethyl, n-propyl or isopropyl, optionally substituted with one or more hydroxyl or amino groups, e.g. CH₂OH, C₂H₄OH, CH₂NH₂ or C₂H₄NH₂. If T is a single atom it is preferably selected from H, F, or Cl.

In another preferred embodiment, all T are the same.

In a preferred embodiment of compounds of formula (VII) and (VIII), R is a residue of an optionally substituted aromatic or non-aromatic 5- to 7-membered carbocyclic or heterocyclic ring like pyridinyl, phenyl, substituted phenyl like benzyl, ethylbenzyl or cyclohexyl. In another preferred embodiment R is selected from a residue of an optionally substituted bicyclical or polycyclic ring like naphthyl or benzimidazolyl. Optional substituents are C₁-C₈-alkyl, hydroxyl, amino or mercapto groups or C₁-C₈-alkyl containing one or more hydroxyl or amino groups like CH₂OH, C₂H₄OH, CH₂NH₂ and/or an oxo-group like CH₂OCH₃ or OC₂H₄OH.

In a more preferred embodiment, R is a residue of a substituted 6-membered aromatic ring, e.g. benzyl or a residue of a 6-membered aromatic ring comprising a methyl or ethyl group like benzyl or ethylphenyl.

In another preferred embodiment, R is attached to the substituted phenyl core via the moiety of formula (IIIb)

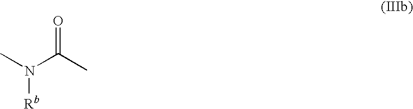

wherein

R^{b} stands for H, C₁-C₈-alkyl, optionally substituted with one or more hydroxyl or amino groups, preferably H or C₁-C₃-alkyl, e.g. methyl, ethyl, n-propyl or isopropyl, more preferably methyl.

In a preferred embodiment of compounds of formula (VII) and (VIII), L is a linker moiety

—CZ¹Z²-CO—N(R^{b})—*

wherein
denotes the attachment of R to said linker moiety; and
Z¹, Z² and R^{b} have the meaning mentioned above.

In a preferred embodiment, Z¹ and Z² are hydrogen or Z¹ is hydrogen and Z² is methyl and R^{b} is H, C₁-C₃-alkyl, e.g. methyl, ethyl, n-propyl or isopropyl, optionally substituted with one or more hydroxyl or amino groups, e.g. CH₂OH, C₂H₄OH, CH₂NH₂ or C₂H₄NH₂.

In a preferred embodiment of compounds of formula (VII) and (VIII), X is selected from residues of DOTA, DTPA, BOPTA, DO3A, HPDO3A, MCTA, DOTMA, DTPA BMA, M4DOTA, M4DO3A, PCTA, TETA, TRITA, HETA, DPDP, EDTA or EDTP.

More preferably, X is selected from residues of DTPA, DOTA, BOPTA, DO3A, HPDO3A, DOTMA, PCTA, DTPA BMA, M4DOTA or M4DO3A. In a most preferred embodiment, X is a chelator of formula (IV).

In a preferred embodiment of compounds of formula (VIII), M is selected from Gd³⁺, Mn²⁺, Fe³⁺, Dy³⁺ and Eu³⁺ with Gd³⁺ being the most preferred paramagnetic ion M.

In a preferred embodiment of compounds of formula (VII) and (VIII) all T are the same, all R are the same, all L are the same, all X are the same and all X′ are the same.

Further preferred compounds of formula (I) and (II) wherein n is 3 are compounds of formula (IX) and (X), consisting of a phenyl core and R-L-X groups attached to said phenyl core, wherein R is a cyclic moiety fused to said phenyl core and groups L-X are attached to R on either carbon atom 1 or 2

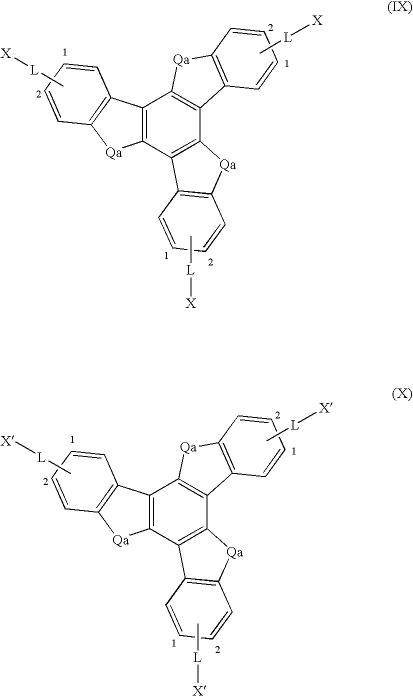

wherein
Qa is the same or different and denotes C(Rc)₂, CH₂S, S, SO, SO₂ or NRc wherein Rc is selected from hydrogen or lower alkyl, preferably C₁-C₃-alkyl, e.g. methyl, ethyl, n-propyl or isopropyl, optionally substituted with one or more hydroxyl or groups or optionally containing one or more oxy groups, e.g. CH₂OH, C₂H₄OH, CH₂OCH₃ or C₂H₄OCH₃;
L may be present or not and if present is the same or different and denotes a linker moiety; and
X and X′ are as defined above.

In a preferred embodiment, Qa is the same and preferably denotes C(Rc)₂, wherein Rc is preferably selected from hydrogen or lower alkyl, preferably C₁-C₃-alkyl, e.g. methyl, ethyl, n-propyl or isopropyl, optionally substituted with one or more hydroxyl or groups or optionally containing one or more oxy groups, e.g. CH₂OH, C₂H₄OH, CH₂OCH₃ or C₂H₄OCH₃;

In a preferred embodiment of compounds of formula (IX) and (X), L is a linker moiety

—CZ¹Z²-CO—N(R^{b})—*

wherein
denotes the attachment of the core to said linker moiety; and
Z¹, Z² and R^{b} have the meaning mentioned above.

In a preferred embodiment, Z¹ and Z² are hydrogen or Z¹ is hydrogen and Z² is methyl and R^{b} is H, C₁-C₃-alkyl, e.g. methyl, ethyl, n-propyl or isopropyl, optionally substituted with one or more hydroxyl or amino groups, e.g. CH₂OH, C₂H₄OH, CH₂NH₂ or C₂H₄NH₂.

In a preferred embodiment of compounds of formula (IX) and (X), X is selected from residues of DOTA, DTPA, BOPTA, DO3A, HPDO3A, MCTA, DOTMA, DTPA BMA, M4DOTA, M4DO3A, PCTA, TETA, TRITA, HETA, DPDP, EDTA or EDTP.

More preferably, X is selected from residues of DTPA, DOTA, BOPTA, DO3A, HPDO3A, DOTMA, PCTA, DTPA BMA, M4DOTA or M4DO3A. In a most preferred embodiment, X is a chelator of formula (IV).

In a preferred embodiment of compounds of formula (X), M is selected from Gd³⁺, Mn²⁺, Fe³⁺, Dy³⁺ and Eu³⁺ with Gd³⁺ being the most preferred paramagnetic ion M.

In a preferred embodiment of compounds of formula (IX) and (X) all L are the same, all X are the same and all X′ are the same.

Further preferred compounds of formula (I) and (II) are compounds of formula (XI) and (XII), consisting of a carbon atom core having n benzene residues R attached to said carbon atom core and n groups -L-X attached to R

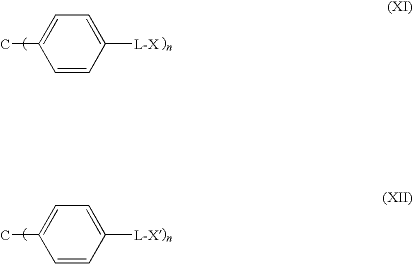

wherein L, X, X′ and n are as defined above.

If n is 3, the forth valence is preferably hydrogen or a group selected from amino, hydroxyl, C₁-C₃-alkyl or halogen, more preferably hydrogen or hydroxyl.

In a preferred embodiment of compounds of formula (XI) and (XII), L is a linker moiety

CZ¹Z²-CO—N(R^{b})—*

wherein
denotes the attachment of R to said linker moiety; and
Z¹, Z² and R^{b} have the meaning mentioned above.

In a preferred embodiment, Z¹ and Z² are hydrogen or Z¹ is hydrogen and Z² is methyl and R^{b} is H, C₁-C₃-alkyl, e.g. methyl, ethyl, n-propyl or isopropyl, optionally substituted with one or more hydroxyl or amino groups, e.g. CH₂OH, C₂H₄OH, CH₂NH₂ or C₂H₄NH₂.

In a preferred embodiment of compounds of formula (XI) and (XII), X is selected from residues of DOTA, DTPA, BOPTA, DO3A, HPDO3A, MCTA, DOTMA, DTPA BMA, M4DOTA, M4DO3A, PCTA, TETA, TRITA, HETA, DPDP, EDTA or EDTP.

More preferably, X is selected from residues of DTPA, DOTA, BOPTA, DO3A, HPDO3A, DOTMA, PCTA, DTPA BMA, M4DOTA or M4DO3A. In a most preferred embodiment, X is a chelator of formula (IV).

In a preferred embodiment of compounds of formula (XII), M is selected from Gd³⁺, Mn²⁺, Fe³⁺, Dy³⁺ and Eu³⁺ with Gd³⁺ being the most preferred paramagnetic ion M.

In a preferred embodiment of compounds of formula (XI) and (XII) all L are the same, all X are the same and all X′ are the same.

Further preferred compounds of formula (I) and (II) wherein n is 3 are compounds of formula (XIII) and (XIV), consisting of a hydroxyl-substituted cyclohexyl core and groups R-L-X attached thereto

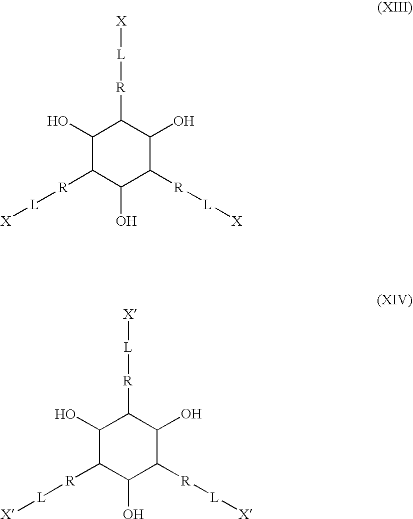

wherein R, L and X are as defined above.

In a preferred embodiment of compounds of formula (XIII) and (XIV), R is a residue of an optionally substituted aromatic or non-aromatic 5- to 7-membered carbocyclic or heterocyclic ring like pyridinyl, phenyl, substituted phenyl like benzyl, ethylbenzyl or cyclohexyl. In another preferred embodiment R is selected from a residue of an optionally substituted bicyclical or polycyclic ring like naphthyl or benzimidazolyl. Optional substituents are C₁-C₈-alkyl, hydroxyl, amino or mercapto groups or C₁-C₈-alkyl containing one or more hydroxyl or amino groups like CH₂OH, C₂H₄OH, CH₂NH₂ and/or an oxo-group like CH₂OCH₃ or OC₂H₄OH.

In a more preferred embodiment, R is a residue of a substituted 6-membered aromatic ring, e.g. benzyl or a residue of a 6-membered aromatic ring comprising a methyl or ethyl group like benzyl or ethylphenyl.

In another preferred embodiment, R is attached to the substituted cyclohexyl core via the moiety of formula (IIIb)

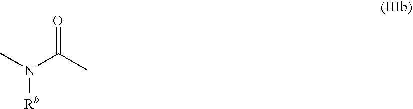

wherein

R^{b} stands for H, C₁-C₈-alkyl, optionally substituted with one or more hydroxyl or amino groups, preferably H or C₁-C₃-alkyl, e.g. methyl, ethyl, n-propyl or isopropyl, more preferably methyl.

In a preferred embodiment of compounds of formula (XIII) and (XIVI), L is a linker moiety

—CZ¹Z²-CO—N(R^{b})—*

wherein
* denotes the attachment of R to said linker moiety; and
Z¹, Z² and R^{b} have the meaning mentioned above.

In a preferred embodiment, Z¹ and Z² are hydrogen or Z¹ is hydrogen and Z² is methyl and R^{b} is H, C₁-C₃-alkyl, e.g. methyl, ethyl, n-propyl or isopropyl, optionally substituted with one or more hydroxyl or amino groups, e.g. CH₂OH, C₂H₄OH, CH₂NH₂ or C₂H₄NH₂

In a preferred embodiment of compounds of formula (XIII) and (XIV), X is selected from residues of DOTA, DTPA, BOPTA, DO3A, HPDO3A, MCTA, DOTMA, DTPA BMA, M4DOTA, M4DO3A, PCTA, TETA, TRITA, HETA, DPDP, EDTA or EDTP.

More preferably, X is selected from residues of DTPA, DOTA, BOPTA, DO3A, HPDO3A, DOTMA, PCTA, DTPA BMA, M4DOTA or M4DO3A. In a most preferred embodiment, X is a chelator of formula (IV).

In a preferred embodiment of compounds of formula (XIV), M is selected from Gd³⁺, Mn²⁺, Fe³⁺, Dy³⁺ and Eu³⁺ with Gd³⁺ being the most preferred paramagnetic ion M.

In a preferred embodiment of compounds of formula (XIII) and (XIV) all R are the same, all L are the same, all X are the same and all X′ are the same.

The compounds of formula (I) and (II) can be synthesized by several synthetic pathways known to the skilled artisan from commercially available starting materials.

Generally, there are two possible pathways: Pathway 1 is based on building blocks and stepwise synthesis while pathway 2 is based on polymerisation of a suitably substituted monomer followed by stepwise synthesis.

For pathway 1, the core is used as a first building block wherein said core is substituted with n reactive groups which allow for the attachment of R. Alternatively, for compounds of formula (IX) and (X) and the compounds of formula (XI) and (XII), the first building block is comprised of the core and R fused or attached to said core and substituted with 3 reactive groups with allow for the attachment of L. Examples of reactive groups are for instance groups with an activated acid functionality, e.g. an acid chloride group or amine groups and methods to introduce these reactive groups said first building block are known in the art. R/L or a precursor of R/L is reacted with the substituted first building block to form a second building block consisting of the core and R or the core, R and L. For this reaction, R/L comprise reactive groups which are able to react with the reactive groups of the first building block to result in the attachment of R/L to said first building block. If compounds of formula (I) or (II) comprise a linker moiety L, said linker moiety is substituted with a reactive group which allows for the attachment to R in the second building block. Likewise, R comprises a reactive group which is able to react with L or a precursor of L to allow for the attachment of L to form a third building block. In a subsequent step, X or X′ or a precursor thereof is attached to the third building block or the second building block in case of compounds of formula (IX) and (X) to form the compounds of formula (I) or (II). If X and/or X′ contain reactive groups like COOH, these groups may need to be protected and suitable protecting groups are known in the art. Alternatively, X is or a precursor thereof is attached to said second or third building block to form the compounds of formula (I) which are then converted into compounds of formula (II) in a subsequent step, which comprises the optional deprotection of X—if used in a protected form—and complex formation with a suitable paramagnetic metal ion M, preferably in the form of its salt (e.g. like Gd(III)acetate or Gd(III)Cl₃).

In another embodiment, a building block consisting of L-X or L-X′ or a precursor thereof is prepared which is then reacted with the second building block or first building block in the case of compounds of formula (IX) and (X) as described above to form the compounds of formula (I) or (II). Again, if X and/or X′ contain reactive groups like COOH, these groups may need to be protected and suitable protecting groups are known in the art. Alternatively, X or a precursor thereof is attached to said second building block to form the compounds of formula (I) which are then converted into compounds of formula (II) in a subsequent step, which comprises the optional deprotection of X—if used in a protected form—and complex formation with a suitable paramagnetic metal ion M, preferably in the form of its salt (e.g. like Gd(III)acetate or Gd(III)Cl₃).

Thus, another aspect of the invention is a method for the preparation of compounds of formula (I) comprising
a) using as a first building block a core A that is substituted with reactive groups which allow for the attachment of R;
b) reacting R or a precursor thereof with said first building block to form a second building block consisting of the core A and R;
c) optionally reacting L or a precursor thereof with said second building block to form a third building block consisting of the core A, R and L; and
d) reacting X or a precursor thereof with said second or third building block.

Yet another aspect of the invention is a method for the preparation of compounds of formula (I) comprising
a) using as a first building block a core A that is substituted with reactive groups which allow for the attachment of R;
b) reacting R or a precursor thereof with said first building block to form a second building block consisting of the core A and R; and
c) reacting a building block consisting of L-X or a precursor thereof with said second building block.

Yet another aspect of the invention a method for the preparation of compounds of formula (I) comprising
a) using a first building block consisting of a core A and R fused or attached to A, wherein R is substituted with reactive groups which allow for the attachment of L or X;
b) optionally reacting L or a precursor thereof with said first building block to form a second building block consisting of the core A, R and L; and
c) reacting X or a precursor thereof with said first or second building block.

Yet another aspect of the invention is a method for the preparation of compounds of formula (I) comprising
a) using a first building block consisting of a core A and R fused or attached to A, wherein R is substituted with reactive groups which allow for the attachment of L; and
b) reacting a building block consisting of L-X or a precursor thereof with said first building block.

The methods of the invention above are suitable for the preparation of compounds of formula (II), if in a subsequent step which comprises the complex formation with a suitable paramagnetic metal ion M, preferably in the form of its salt (e.g. like Gd(III)acetate or Gd(III)Cl₃).

For pathway 2, a suitably substituted monomer is polymerised, i.e. by trimerisation a trimer (n is 3) or by tetramerisation an intermediate in the form of a tetramer (n is 4) is synthesized from said monomer and said polymerisation is followed by stepwise synthesis. Suitably, the monomer comprises a moiety, which, upon polymerisation forms A. Further, the monomer comprises R comprising a reactive moiety or a precursor thereof which allows for the attachment of L or a precursor thereof, if present or X/X′ or a precursor thereof. An example of such a reactive moiety may be an amino group and a precursor thereof may be a nitro group which in itself is not reactive, i.e. the nitro group would not react in the polymerisation reaction. After polymerisation, the nitro group may be reduced to a reactive amino group. Other reactive groups or precursor thereof, e.g. a carboxyl group and an ester as a possible precursor thereof are known in the art. After polymerisation and optional conversion of a precursor into a reactive group, L or a precursor of L—if present in the final reaction product, is reacted with the intermediate obtained by tri or tetramerisation of the monomer. In a subsequent step, X or X′ is attached to form the compounds of formula (I) or (II). If X and/or X′ contain reactive groups like COOH, these groups may need to be protected and suitable protecting groups are known in the art. Alternatively, X is attached to form the compounds of formula (I) which are then converted into compounds of formula (II) in a subsequent step, which comprises the optional deprotection of X—if used in a protected form—and complex formation with a suitable paramagnetic metal ion M, preferably in the form of its salt (e.g. like Gd(III)acetate or Gd(III)Cl₃).

Thus, another aspect of the invention is a method for the preparation of compounds of formula (I) comprising
a) trimerisation or tetramerisation of a monomer comprising a moiety, which, upon trimerisation or tetramerisation forms A, said monomer further comprises R comprising a reactive moiety or a precursor thereof which allows for the attachment of L or a precursor thereof, if L is present, or X or a precursor thereof to form an intermediate;
b) optionally reacting L or a precursor thereof with said intermediate; and
c) reacting X or a precursor thereof with the intermediate or the reaction product of step b).

The method of the invention above is suitable for the preparation of compounds of formula (II), if in a subsequent step which comprises the complex formation with a suitable paramagnetic metal ion M, preferably in the form of its salt (e.g. like Gd(III)acetate or Gd(III)Cl₃).

Compounds of formula (V) and (VI) may be synthesized by either pathway 1 or 2, preferably by pathway 2. The first step of said pathway 2 is the trimerisation of 4-nitrophenylisocyanate or a derivative thereof like 2-methyl-4-nitrophenylisocyanate 1 to result in an intermediate comprising a cyanuric acid core and a benzene residue or substituted benzene residue R, wherein R comprises a nitro group 2 (1,3,5-tris-(4-nitro-2-methylphenyl)-[1,3,5]triazinane-2,4,6-trione) which is the precursor of a reactive group (an amino group) which allows for the attachment of L or X/X′ 3 (1,3,5-tris-(4-amino-2-methylphenyl)-[1,3,5]triazinane-2,4,6-trione).

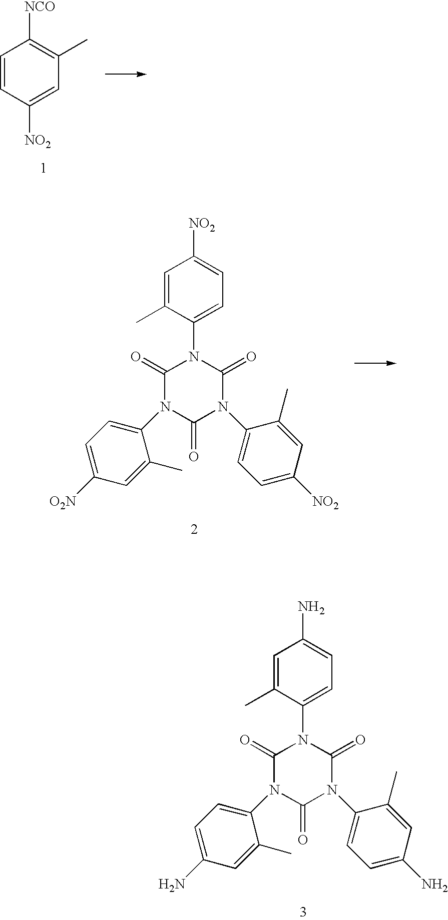

The starting compound 1 can be obtained by reaction of 2-methyl-4-nitroaniline with phosgene. By carrying out the trimerisation in a sealed vessel better yields are obtained. Further, by carrying out the hydrogenation to obtain 3 in a solvent mixture of tetrahydrofuran (THF) and water, shorter reaction times and higher yields could be achieved.

Thus, in another aspect the invention provides an improved method for producing 1,3,5-tris-(4-amino-2-methylphenyl)-[1,3,5]triazinane-2,4,6-trione) by trimerisation of 2-methyl-4-nitrophenylisocyanate in a sealed vessel and hydrogenation of the trimer obtained in a solvent mixture of tetrahydrofuran and water. Preferably, hydrogenation is carried out with Pd/C as catalyst.

The attachment of L to the amino groups of 3 may be carried out as known in the art. In a preferred embodiment, L is a linker moiety comprising a residue of an N-heterocycle, preferably a triazole residue, a pyrazinone residue or an imidazole residue.

If L comprises an imidazole residue, the intermediate 3 is preferably reacted with a tosylmethyl isocyanide reagent as described in J. Sisiko et al., J. Org. Chem. 2000, 65, 1516-1524 to result in 4:

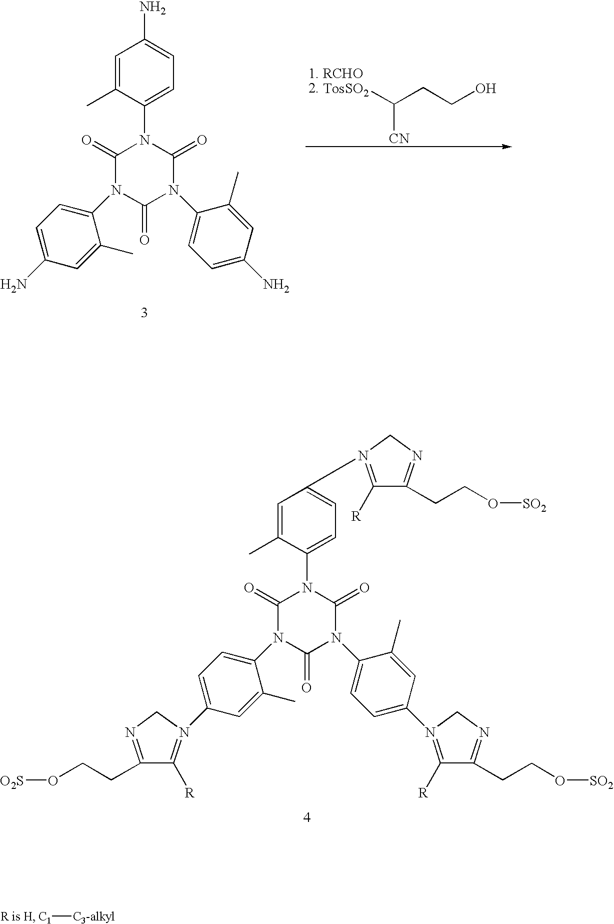

The reaction product 4 contains mesylate groups that readily react with X, e.g. tert-butyl protected DO3A to result in a compound of formula (I), which may be converted into a compound of formula (II) in a subsequent step, wherein said subsequent step comprises the deprotection of X and complex formation with a suitable paramagnetic metal ion M, preferably in the form of its salt (e.g. like Gd(III)acetate or Gd(III)Cl₃).

If L comprises a pyrazinone residue, the intermediate 3 is preferably reacted with a diketone and a serine derivative as described in Chuyen et al., Agr. Biol. Chem. 37(2), 1973, 327-334 to result in 5:

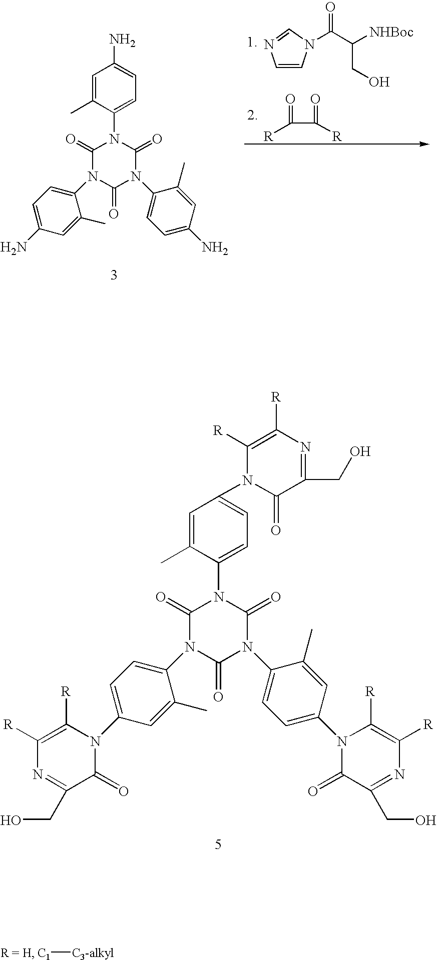

The reaction product 5 contains hydroxyl groups which easily might get converted into a mesylate groups that readily react with X, e.g. tert-butyl protected DO3A, to result in a compound of formula (I), which may be converted into a compound of formula (II) in a subsequent step, wherein said subsequent step comprises the deprotection of X and complex formation with a suitable paramagnetic metal ion M, preferably in the form of its salt (e.g. like Gd(III)acetate or Gd(III)Cl₃).

If L comprises a triazole residue, such a triazole ring is easily accessible by the Cu(I)-catalyzed cyclization of an organic azide and a terminal acetylene, as described in Vsevolod et al., Angew Chem. Int. Ed. 2002, 41(14), 1596-1599. The handling of organic azides is however troublesome, especially at larger scales, since they may decompose violently. Thus, in a preferred embodiment, the intermediate 3 is in a one pot reaction converted to an azide using standard diazotation conditions followed by addition of sodium azide. Upon completion of the reaction, the reaction mixture is neutralized and propargylic alcohol is added together with a Cu(I) source to result in 6:

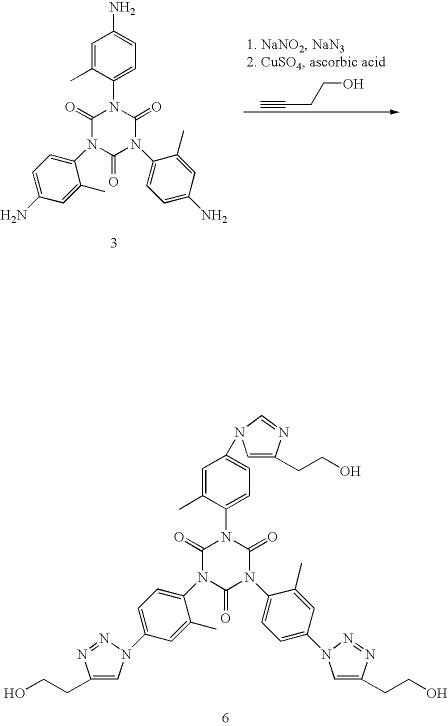

The reaction product 6 contains hydroxyl groups which easily might get converted into a mesylate groups that readily react with X, e.g. tert-butyl protected DO3A, to result in a compound of formula (I), which may be converted into a compound of formula (II) in a subsequent step, wherein said subsequent step comprises the deprotection of X and complex formation with a suitable paramagnetic metal ion M, preferably in the form of its salt (e.g. like Gd(III)acetate or Gd(III)Cl₃).

Compounds of formula (VII) and (VIII) may be synthesized by either pathway 1 or 2. For pathway 1, the first step is the synthesis of a first building block, i.e. a core being substituted with 3 reactive groups which allow for the attachment of R.

If T is hydrogen, 1,3,5-benzenetricarboxylic acid (trimesic acid), a commercially available compound may be used as a starting compound. Trimesic acid may be converted into the acid chloride by methods known in the art, e.g. by reaction with PCl₅. The 1,3,5-benzenetricarboxylic acid chloride is the first building block and consists of an unsubstituted phenyl core being substituted with 3 reactive groups, i.e. the carboxylic acid chloride groups. Said groups can be reacted with groups R which for instance contain an amino group and thus form a —CO—NH— group upon reaction with the carboxylic acid chloride groups.

If T is C₁-C₃-alkyl, e.g. methyl the first building block may be synthesized using 1,3,5-tri-C₁-C₃-alkylbenzene, e.g. 1,3,5-trimethylbenzene or 1,3,5-triisopropylbenzene as a starting compound and proceeding as described in Examples 4-7.

If T is a halogen, for instance Cl, the first building block may be synthesized using 1,3,5-trimethylbenzene as a starting compound and converting said starting compound to 1,3,5-trimethyl-2,4,6-trichlorobenzene as described in K. Shoji et al., Bull. Chem. Soc. Jpn. 62, 1989, 2096-2098. Subsequent oxidation results in 2,4,6-trichlorobenzene-1,3,5-tricarboxylic acid which may be converted to a reactive acid chloride by reaction with thionylchloride.

The first step of pathway 2 for the synthesis of compounds of the formula (VII) and (VIII) is the trimerisation of a monomer R—C(O)CH₃ in the presence of triflic acid which results in an intermediate consisting of the phenyl core with substituents R. An example of such a monomer is 4-acetamido-2-methylacetophenone and the trimerisation of said monomer results in an intermediate 7,

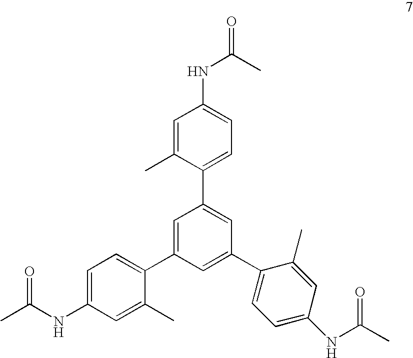

consisting of a phenyl core, and R which is a residue of toluene, wherein R is substituted with a reactive group —NH—CO—CH₃ which can be converted to a linker or precursor of a linker to which X/X′ is attached. The synthesis is described in detail in Example 2. Compounds of formula (IX) and (X) can be synthesized by either pathway 1 or 2. According to pathway 2, preferably those compounds of formula (IX) and (X) are synthesised wherein Qa is CH₂ or C(Rc)₂, wherein Rc is lower alkyl, optionally substituted with one or more hydroxyl groups or optionally containing one or more oxy groups or wherein Qa is S, SO or SO₂.

If Qa is CH₂ or C(Rc)₂, wherein Rc is lower alkyl, optionally substituted with one or more hydroxyl groups or optionally containing one or more oxy groups, the compounds of formula (IX) or (X) can be easily synthesized by acid catalyzed timerisation of 2-indanone or 1-(Rc)₂-2-indanone.

If Qa is S, the compounds of formula (IX) or (X) may be synthesized by trimerisation of 3(2H)benzothiophenone as described by Dagliesh et al., J. Chem. Soc 910 (1945) and Proetzsch et al., Z. Naturforsch. 31B, 529 (1976).

If Qa is SO or SO₂, the compounds of formula (IX) or (X) may be synthesized as described in the previous paragraph and S can be oxidised by methods known in the art. The oxidation also increases the solubility of the intermediates obtained by said trimerisation.

Since the trimerisation of 3(2H)benzothiophenone leads to intermediates which are poorly soluble, it is preferred to increase solubility at an early stage in the synthesis. A possible way is shown in reaction scheme 1:

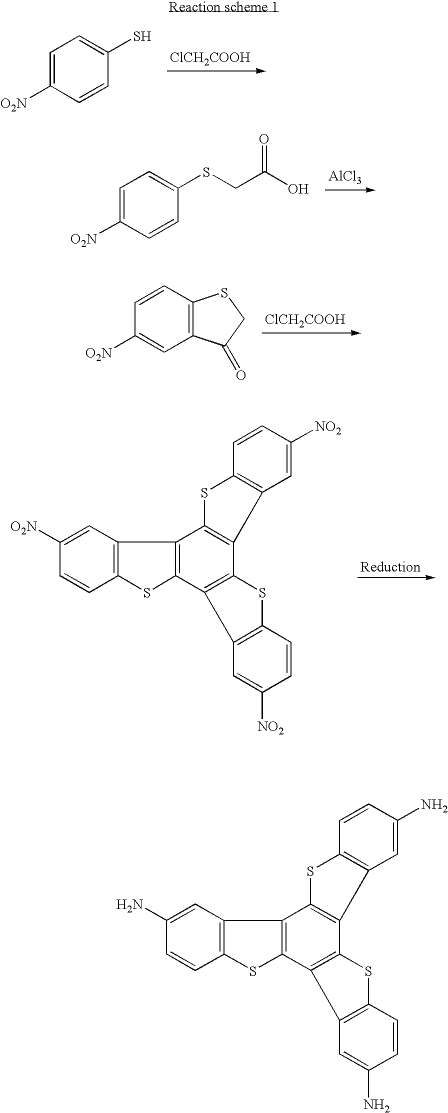

By choosing this approach, reactive groups, i.e. amino groups are already introduced into the molecule which then can be used for the attachment of L or X, if L is not present, to the C-atom 1.

If Qa is NRc, the compounds of formula (IX) and (X) may be synthesized by Ullman coupling of N-substituted 2-iodoindole, e.g. 2-iodo-N-methylindole if Rc is methyl, as described by Bergman et al., Tetrahedron 36, 1439 (1980).

After having synthesized the intermediate, said intermediate is activated by introducing suitable reactive groups at the C-atom 1 or 2, depending on where groups -L-X are to be attached. To activate the intermediate at the C-atom 1, the intermediate is conveniently synthesized by trimerisation of a compound which already includes said reactive groups, e.g. by trimerisation of a molecule containing a nitro group and reduction of said nitro group as shown in reaction scheme 1 or by trimerisation of a molecule containing a bromo-group, e.g. trimerisation of 6-bromo-1-indanone to obtain a truxene intermediate containing a reactive bromo-group at C-atom 1 (see Gomez-Lor et al., Eur. J. Org. Chem. 2001, 2107-2114). To activate the intermediate at C-atom 2, said intermediate may be reacted with molecular bromine whereby an activated intermediate is obtained comprising reactive bromo-groups at C-atom 2 as described by Gomez-Lor et al., Eur. J. Org. Chem. 2001, 2107-2114. Alternatively, the intermediate may be nitrated and the nitro groups reduced to result in an activated intermediate with reactive amino-groups at C-atom 2.

If compounds of formula (IX) and (X) comprise a linker moiety L, said linker moiety is substituted with a reactive group which allows for the attachment to the intermediate. L or a precursor of L is reacted with said intermediate by methods known in the art. In a subsequent step, X or X′ is attached to form the compounds of formula (IX) or (X)

In another embodiment, a building block consisting of L-X or L-X′ is prepared which is then reacted with the intermediate described above to form the compounds of formula (IX) or (X). If X and/or X′ contain groups like COOH, these groups may need to be protected. Suitable protecting groups are known in the art. X can be converted into X′ by an optional deprotection reaction and complex formation with a suitable paramagnetic metal ion M, preferably in the form of its salt (e.g. like Gd(III)acetate or Gd(III)Cl₃).

Compounds of formula (XI) and (XII) can be synthesized according to pathway 1. The first building block, i.e. the core having attached thereto n groups R wherein said groups R comprise reactive amino groups may either be synthesized as described in L. M. Werbel et al., J. Org. Chem. 29, 1964, 967-968 or is commercially available. Briefly, the first building block may be synthesized as follows:

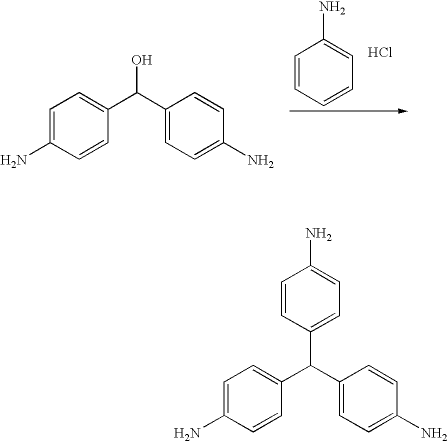

If compounds of formula (XI) and (XII) comprise a linker moiety L, said linker moiety is substituted with a reactive group which allows for the attachment to the first building block. L or a precursor of L is reacted with said first building block by methods known in the art. In a subsequent step, X or X′ is attached to form the compounds of formula (XI) or (XII)

In another embodiment, a building block consisting of L-X or L-X′ is prepared which is then reacted with the first building block described above to form the compounds of formula (XI) or (XII). If X and/or X′ contain groups like COOH, these groups may need to be protected. Suitable protecting groups are known in the art. X can be converted into X′ by an optional deprotection reaction and complex formation with a suitable paramagnetic metal ion M, preferably in the form of its salt (e.g. like Gd(III)acetate or Gd(III)Cl₃).

Compounds of formula (XIII) and (XIV) can be synthesized according to pathway 1. The first building block i.e. the hydroxyl-substituted cyclohexyl core having attached thereto 3 reactive groups, e.g. amino groups may be synthesized as follows:

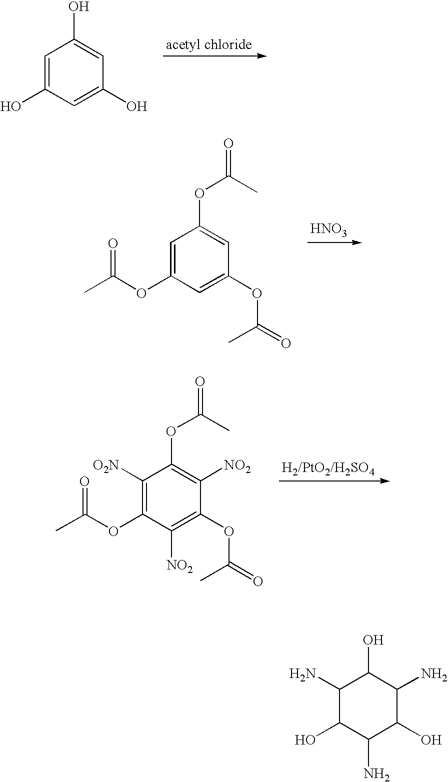

R^{X} or a reactive precursor of R is reacted with the first building block above to form a second building block consisting of the hydroxyl-substituted cyclohexyl core and R. If compounds of formula (XIII) and (XIV) comprise a linker moiety L, said linker moiety is substituted with a reactive group which allows for the attachment to the second building block. L or a precursor of L is reacted with said second building block by methods known in the art. In a subsequent step, X or X′ is attached to form the compounds of formula (XIII) or (XIV)

In another embodiment, a building block consisting of L-X or L-X′ is prepared which is then reacted with the second building block described above to form the compounds of formula (XIII) or (XIV). If X and/or X′ contain groups like COOH, these groups may need to be protected. Suitable protecting groups are known in the art. X can be converted into X′ by an optional deprotection reaction and complex formation with a suitable paramagnetic metal ion M, preferably in the form of its salt (e.g. like Gd(III)acetate or Gd(III)Cl₃).

The invention is illustrated by the examples in the corresponding section of this patent application.

The compounds of formula (II) and preferred embodiments thereof may be used as MR contrast agents. For this purpose, the compounds of formula (II) are formulated with conventional physiologically tolerable carriers like aqueous carriers, e.g. water and buffer solution and optionally excipients.

Hence in a further aspect the present invention provides a composition comprising a compound of formula (II) or preferred embodiments thereof and at least one physiologically tolerable carrier.

In a further aspect the invention provides a composition comprising a compound of formula (II) and preferred embodiments thereof and at least one physiologically tolerable carrier for use as MR imaging agent or MR spectroscopy agent.

To be used as agents for MR imaging or spectroscopy of the human or non-human animal body, said compositions need to be suitable for administration to said body. Suitably, the compounds of formula (II) or preferred embodiments thereof and optionally pharmaceutically acceptable excipients and additives may be suspended or dissolved in at least one physiologically tolerable carrier, e.g. water or buffer solutions. Suitable additives include for example physiologically compatible buffers like tromethamine hydrochloride, chelators such as DTPA, DTPA-BMA or compounds of formula (I) or preferred embodiments thereof, weak complexes of physiologically tolerable ions such as calcium chelates, e.g. calcium DTPA, CaNaDTPA-BMA, compounds of formula (I) or preferred embodiments thereof wherein X forms a complex with Ca²⁺ or CaNa salts of compounds of formula (I) or preferred embodiments thereof, calcium or sodium salts like calcium chloride, calcium ascorbate, calcium gluconate or calcium lactate. Excipients and additives are further described in e.g. WO-A-90/03804, EP-A-463644, EP-A-258616 and U.S. Pat. No. 5,876,695, the content of which are incorporated herein by reference.

Another aspect of the invention is the use of a composition comprising a compound of formula (II) or preferred embodiments thereof and at least one physiologically tolerable carrier as MR imaging agent or MR spectroscopy agent.

Yet another aspect of the invention is a method of MR imaging and/or MR spectroscopy wherein a composition comprising a compound of formula (II) or preferred embodiments thereof and at least one physiologically tolerable carrier is administered to a subject and the subject is subjected to an MR procedure wherein MR signals are detected from the subject or parts of the subject into which the composition distributes and optionally MR images and/or MR spectra are generated from the detected signals.

In a preferred embodiment, the subject is a living human or non-human animal body.

In a further preferred embodiment, the composition is administered in an amount which is contrast-enhancing effective, i.e. an amount which is suitable to enhance the contrast in the MR procedure.

In a preferred embodiment, the subject is a living human or non-human animal being and the method of MR imaging and/or MR spectroscopy is a method of MR angiography, more preferred a method of MR peripheral angiography, renal angiography, supra aortic angiography, intercranial angiography or pulmonary angiography.

In another preferred embodiment, the subject is a living human being or living non-human animal being and the method of MR imaging and/or MR spectroscopy is a method of MR tumour detection or a method of tumour delineation imaging.

In another aspect, the invention provides a method of MR imaging and/or MR spectroscopy wherein a subject which had been previously administered with a composition comprising a compound of formula (II) or preferred embodiments thereof and at least one physiologically tolerable carrier is subjected to an MR procedure wherein MR signals are detected from the subject or parts of the subject into which the composition distributes and optionally MR images and/or MR spectra are generated from the detected signals.

The term “previously been administered” means that the method as described above does not contain an administration step of said composition to said subject. The administration of the composition has been carried out previous to the method as described above, i.e. before the method of MR imaging and/or MR spectroscopy according to the invention is commenced.

### EXAMPLES

### Example 1

### Preparation of 1,3,5-tris-(N-(DO3A-acetamido)-N-methyl-4-amino-2-methyl-phenyl)-[1,3,5]triazinane-2,4,6-trione (22), a compound of formula (I) and a gadolinium derivative thereof (23), a compound of formula (II)

### 1a) Preparation of 2-methyl-4-nitrophenylisocyanate (14)

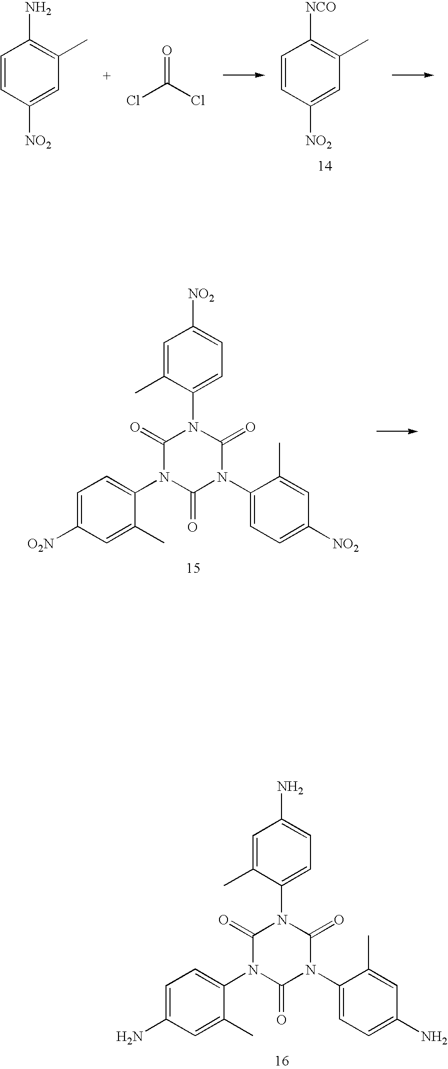

2-Methyl-4-nitroaniline (35.0 g, 230 mmol) was dissolved in ethyl acetate (400 ml) and cooled to 0° C. Phosgene (180 ml, 20% in toluene) was added drop wise over 30 min, precipitation of a white salt followed instantly. After the last addition the temperature was allowed to slowly rise to room temperature, and then the reaction mixture was brought to reflux (about 100° C.). It was refluxed for 2 h 30 min, after which 200 ml of solvent was distilled off before the temperature was lowered to 80° C. and phosgene (140 ml, 20% in toluene) was added drop wise. After the last addition the reaction solution was refluxed for 3 hours, allowed to cool to room temperature and concentrated to dryness. The brown/yellow material was dissolved in diethyl ether (250 ml), filtered and concentrated to give a pale brown powder (36 g, 88%).

### 1b) Preparation of 1,3,5-tris-(4-nitro-2-methyl-phenyl-[1,3,5]triazinane-2,4,6-trione (15)

To 2-Methyl-4-nitrophenylisocyanate (36.0 g) in a 250 ml flask was added DMSO (50 ml) and the flask was sealed with a glass stopper which was kept in place with a plastic clip. The flask was immediately lowered into an oil bath heated to 85° C. and the dark brown reaction solution was heated for 16 h 30 min. The oil bath was removed and the reaction solution was allowed to cool to room temperature before being poured into water (800 ml), sonicated, and the precipitate was filtered off. The filter cake was added to ethanol (500 ml) and was refluxed for 4 hours, then allowed to cool to room temperature and the product was filtered off to give an off-white powder (28.1 g, 78%).

### 1c) Preparation of 1,3,5-tris-(4-amino-2-methyl-phenyl)-[1,3,5]triazinane-2,4,6-trione (16)

1,3,5-Tris-(4-nitro-2-methyl-phenyl)-[1,3,5]triazinane-2,4,6-trione (2.86 g, 5.4 mmol) was dissolved in THF (70 ml). HCl (4.5 ml, 6M), H2O (18 ml) and Pd/C (0.6 g, 10%) was added. The reaction vessel was evacuated and filled with argon in three cycles before hydrogenated on a Parr hydrogenation apparatus (60 psi). After 2 hours the excess hydrogen was evacuated with a membrane pump and the Pd/C (10%) was filtered off. The clear reaction solution was concentrated until no more THF remained and the pH adjusted to 7 with NaHCO₃ (˜3.7 g). The aqueous phase was extracted with ethyl acetate (3×100 ml) and the combined organic phases were dried with MgSO₄, filtered and concentrated to give a brown powder. The crude was re-crystallised from methanol to give the product as an off-white powder (1.9 g, 80%).

### 1d) Preparation of 1,3,5-tris-(4-formamido-2-methyl-phenyl)-[1,3,5]triazinane-2,4,6-trione (17)

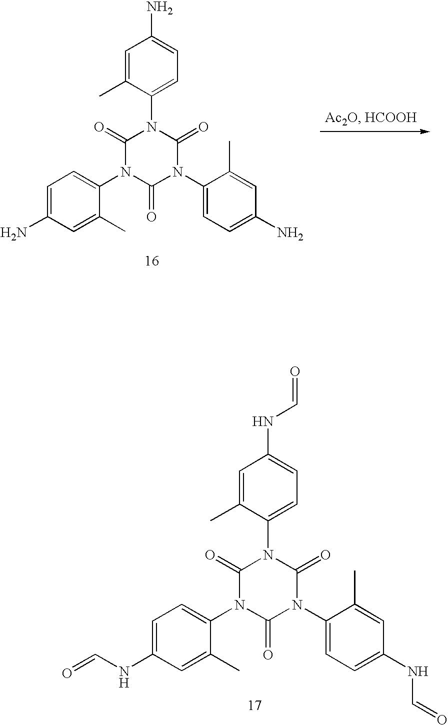

Formic acid (175 mL) was put in an ice-cooled 500 mL round-bottom flask. Acetic anhydride (15 mL, 0.16 mol) was added and the yellow solution was stirred under argon for 1 h at 0° C. The triamine 16 (8.7 g, 0.020 mol) was added to this solution and the ice bath was removed. After stirring under argon at room temperature for 30 minutes HPLC showed complete reaction. The solvent was removed in vacuo and the brown, sticky residue was suspended in H₂O and filtered off. It was then washed thoroughly with H₂O to make sure all acid was removed. The product was a pale-brown solid (10.2 g, 99%).

### 1e) Preparation of 1,3,5-tris-(N-formyl-N-methyl-4-amino-2-methyl-phenyl)-[1,3,5]-triazinane-2,4,6-trione (18)

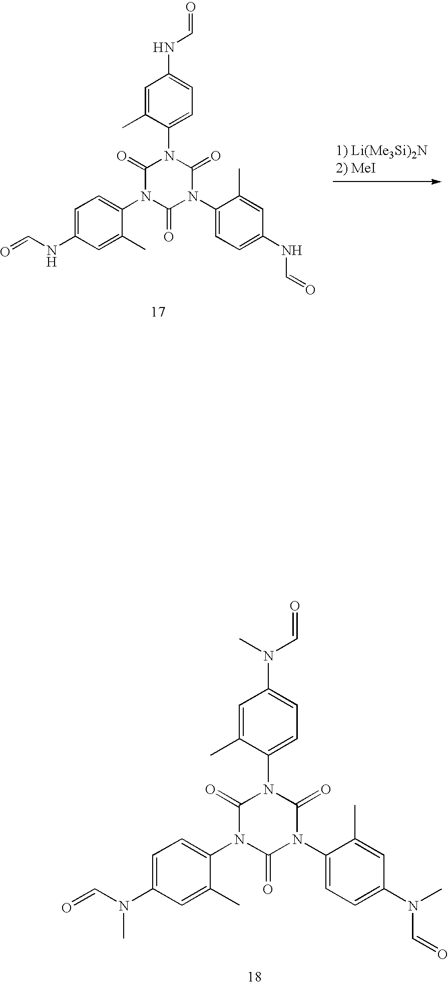

All glassware was carefully dried in oven and DMF was dried over 4 Å molecular sieves.

Li(Me₃Si)₂N (116 mL, 0.116 mol, 1 M in hexane) was added to a DMF-solution (115 mL) of 17 (10.2 g, 0.0193 mol) in 500 mL round-bottom flask. The reaction mixture, which turned from a light brown solution to a brick-red slurry, was stirred under argon for 1 h. Methyl iodide (12.2 mL, 0.196 mol) was added and the reaction mixture was stirred for 2 h or until complete methylation could be shown on HPLC. The hexane was then removed on rotary evaporator and the residue was poured into a solution of NaH₂PO₄ (1300 mL, 100 mM) under vigorous stirring. The precipitate of 18 formed was filtered off as a pale solid (6.7 g, 60%).

### 1f) Preparation of 1,3,5-tris-(N-methyl-4-amino-2-methyl-phenyl)-[1,3,5]triazinane-2,4,6-trione (19)

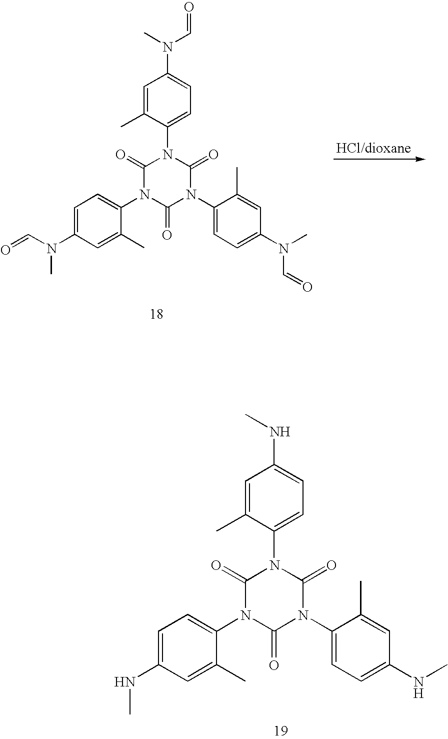

Dioxane (52 mL), HCl (52 mL, 6 M) and 18 (6.5 g, 11 mmol) were mixed in a 250 mL round-bottom flask to form a pale slurry. The reaction mixture was heated to reflux for 30 minutes under argon. The now yellow solution was allowed to cool to room temperature and solvents were then removed on a rotary evaporator. The orange residue was then dissolved in 500 mL H₂O and neutralized with a solution of NaHCO₃ (sat.) under vigorous stirring. The precipitate formed was filtered off and washed several times with H₂O giving a pale solid (4.7 g, 84%).

### 1g) Preparation of 1,3,5-tris-(N-chloroacetyl-N-methyl-4-amino-2-methyl-phenyl)-[1,3,5]triazinane-2,4,6-trione (20)

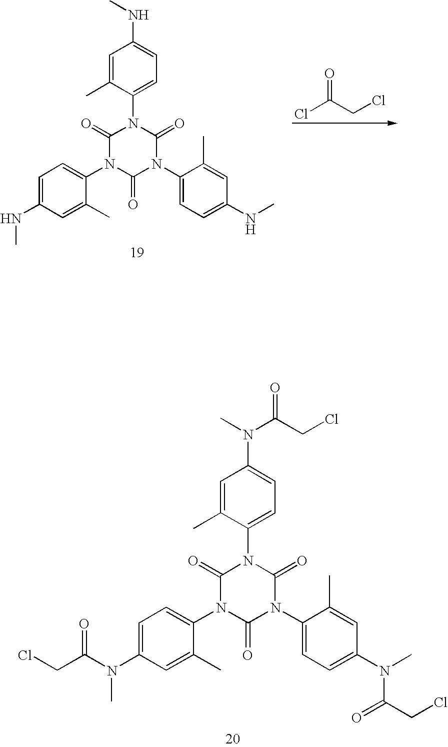

In a 100 mL round-bottom flask 19 (4.6 g, 9.5 mmol) was dissolved in DMA (15 mL) and chloroacetyl chloride (2.6 mL, 33 mmol) was added under stirring at 0° C. The reaction was stirred under argon at r t for 30 min or until HPLC showed complete chloroacetylation. The slurry was then poured into a large beaker with water (500 mL) under vigorous mechanical stirring. The precipitate formed was filtered off and dried in vacuo at 0.3 mbar (6.3 g). The pale solid was dissolved in 70 mL acetonitrile and poured into 500 mL H₂O under vigorous mechanical stirring. The precipitate formed was filtered off and left to dry in a desiccator (6.1 g, 89%).

### 1h) Preparation of 1,3,5-tris-(N-(DO3A t-butylester-acetamido)-N-methyl-4-amino-2-methyl-phenyl)-[1,3,5]triazinane-2,4,6-trione (21)

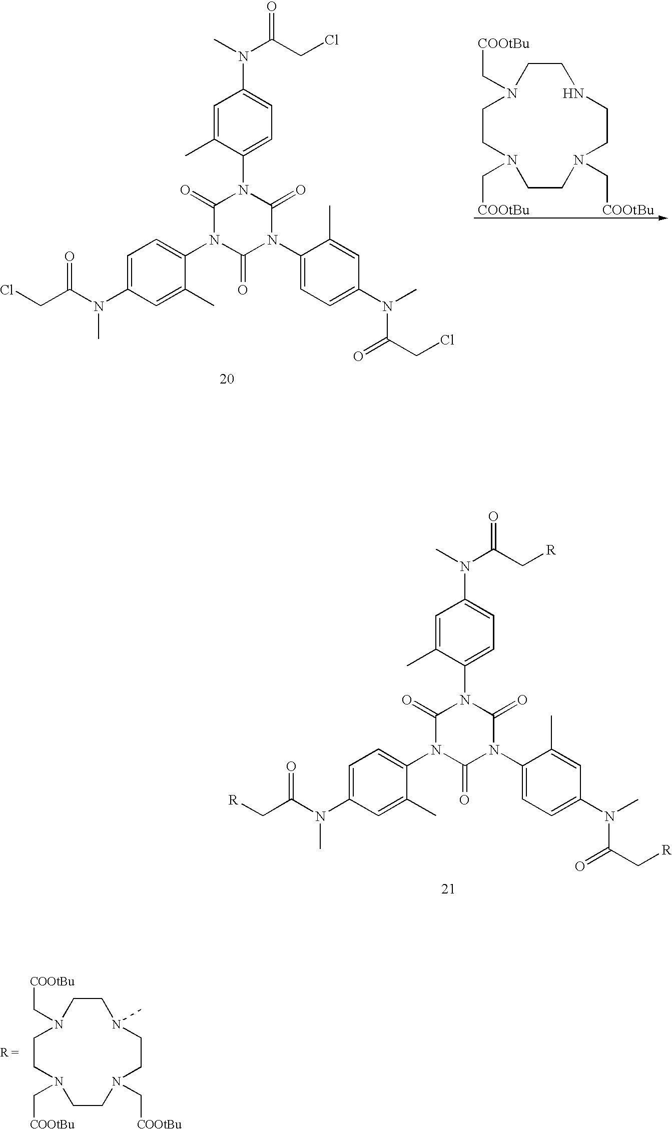

In a 50 mL round-bottom flask, 20 (0.50 g, 0.70 mmol) was suspended together with DO3A t-butyl ester (2.5 g, 4.2 mmol), diisopropylethylamine (910 μl, 5.2 mmol) and acetonitrile (15 mL). After sonication the reaction mixture was stirred at 75° C. under argon until LC/MS showed complete coupling. The solvents were then removed on rotary evaporator and the crude product (2.9 g) was used in the subsequent reaction.

### 1i) Preparation of 1,3,5-tris-(N-(DO3A-acetamido)-N-methyl-4-amino-2-methyl-phenyl)-[1,3,5]triazinane-2,4,6-trione (22)

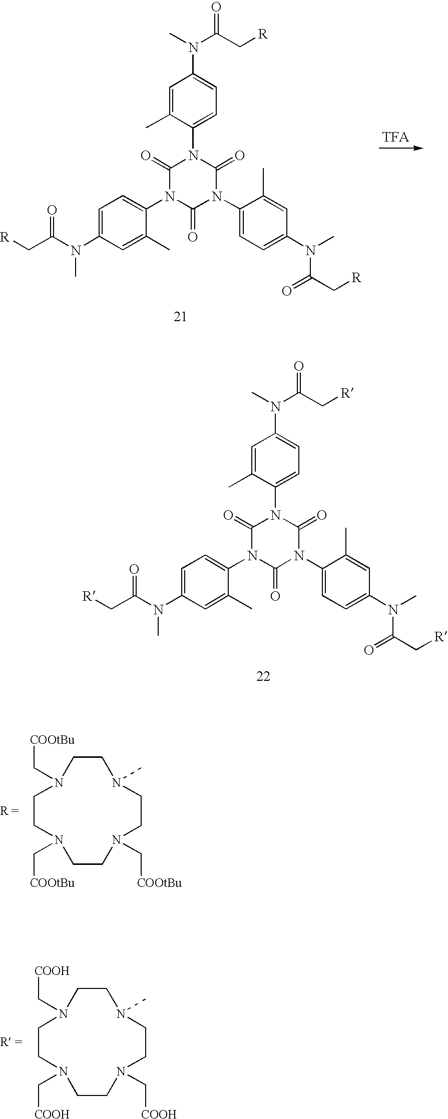

The crude product of 21 (1.9 g) was dissolved in TFA (130 mL) and CH₂Cl₂ (130 mL) and was stirred at 50° C. under argon. The solution was stirred for 1 h or until LC/MS showed complete deprotection. The solvents were then removed on rotary evaporator and the residue was dried in vacuo overnight. The crude product (2.4 g) was then used in the subsequent step.

### 1j) Preparation of a gadolinium derivative of 1,3,5-tris-(N-(DO3A-acetamido)-N-methyl-4-amino-2-methyl-phenyl)-[1,3,5]triazinane-2,4,6-trione (23)

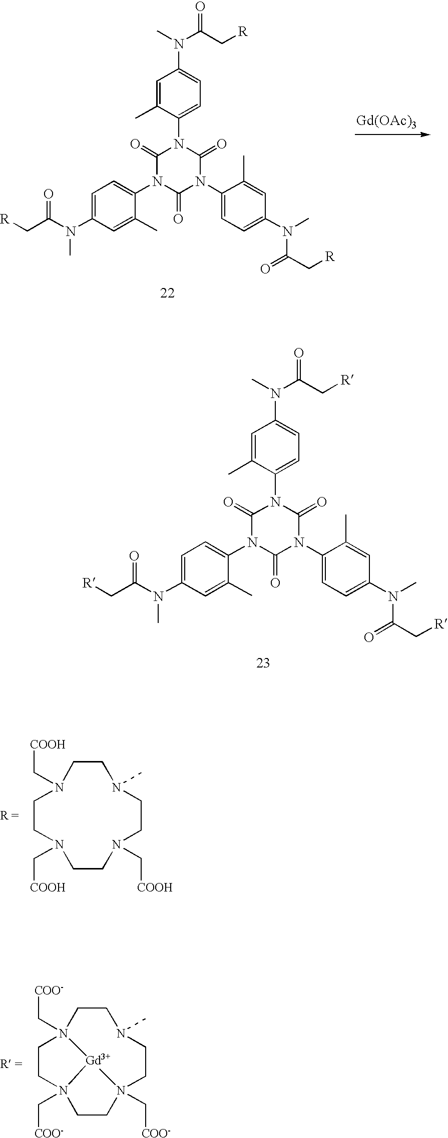

The crude product of 22 (2.4 g) was dissolved in water and Gd(OAc)₃ (1.4 g, 4.2 mmol) was added under stirring. Vacuum (0.3 mbar) was then put on and the reaction was monitored continuously by LC/MS. When complete complexation was detected, the solvents were removed in vacuo. The crude product of 3.1 g was then purified by preparative HPLC (410 mg, 42% from 20).

Compound 23 was dissolved in human blood plasma and the longitudinal relaxivity rl was measured at 37° C. at the following fields:

0.25 T, an rl of 10.7 was measured; and
1.5 T, an rl of 11.6 mM¹s¹ was measured; and
2.35 T, an rl of 10.1 mM¹s¹ was measured; and
3 T, an rl of 9.9 mM¹s¹ was measured.

Compared to other MR contrast agent compounds known in the art, the rl at 3 T of the compound according to the invention shown above is much higher. rl values for other MRI contrast agents are at 3 T in human blood plasma at 37° C. (data published in Invest Radiol 2006, Vol. 41, 213-221):

Multihance™: rl is 6.3 mM¹s¹
Magnevist™: rl is 3.3 mM¹s¹

### Example 2

### Preparation of 1,3,5-tris-(N-(DO3A-acetamido)-N-methyl-4-amino-2-methyl-phenyl)-benzene (6), a compound of formula (I) and a gadolinium derivative thereof (7), a compound of formula (II)

### 2a) Preparation of 1,3,5-tris-(4-acetamido-2-methyl-phenyl)-benzene (1)

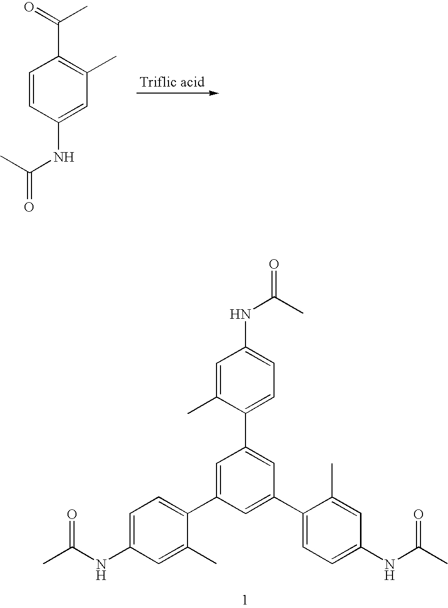

4-Acetamido-2-methylacetophenone (Aldrich, 5.0 g, 26.1 mmol) was melted at 180° C. in an open round bottled flask. To the stirred homogenous solution was added triflic acid (Fluka, 250 μl, 2.9 mmol). After 1 h another 250 μl of triflic acid were added. The thick brown mixture was allowed to cool after 5 h. The product was purified by preparative HPLC and obtained in 570 mg after lyophilisation, 4% yield. The structure was confirmed by NMR analysis.

### 2b) Preparation of 1,3,5-tris-(4-acetamido-N-methyl-4-amino-2-methyl-1-phenyl)-benzene

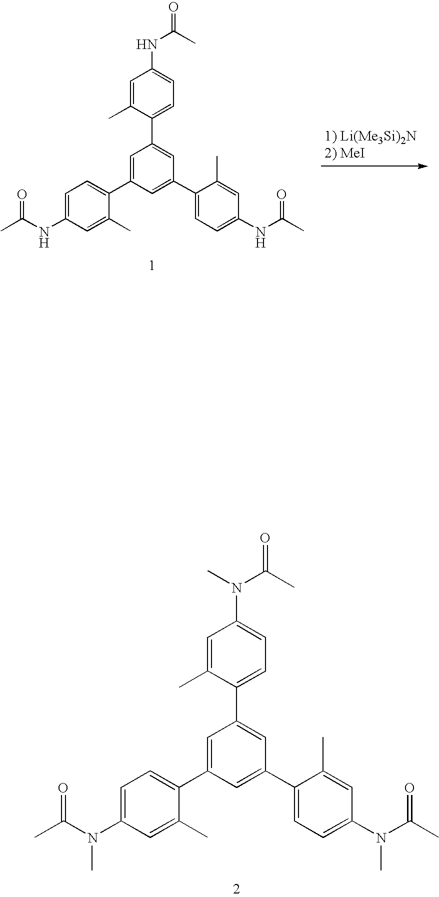

To a solution of 1 (654 mg, 1.259 mmol) in dry DMF (20 ml) was added lithium bis(trimethylsilyl)amide (Aldrich, 7.56 ml, 7.56 mmol). The reaction mixture, which turned from a transparent brown solution to thick brown slurry, was stirred under argon for 1 h. Methyl iodide (Fluka, 0.956 ml, 15.36 mmol) was added, the solution got clear, and aluminium foil was wrapped around the round bottled flask to prevent light exposure. The reaction was completed after 2 hours. The solvent was evaporated (rotary evaporator). The product mixture was dissolved in ethyl acetate and washed with water. The organic phase was dried (Na₂SO₄) and evaporated giving 700 mg, 99% yield. The structure was confirmed by LC-MS.

### 2c) Preparation of 1,3,5-tris-(N-methyl-4-amino-2-methyl-phenyl)-benzene (3)

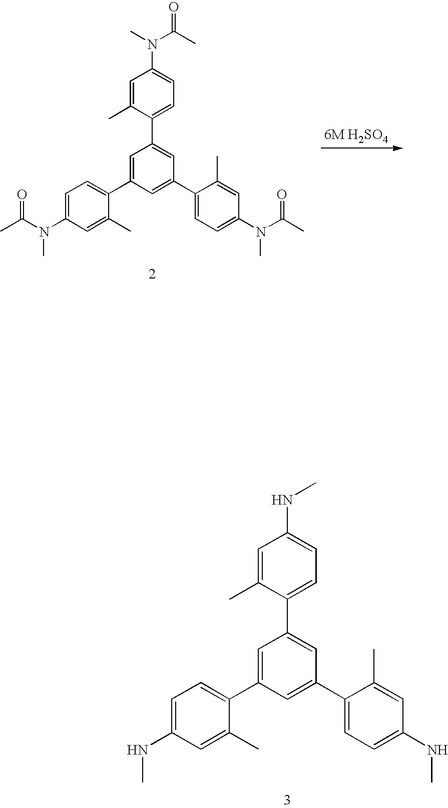

A mixture of 2 (700 mg, 1.246 mmol) and 6 M H₂SO₄ (80 ml) was heated by microwave irradiation at 120° C. for 30 min. The acid was neutralized with saturated NaHCO₃ under vigorous stirring. The precipitate formed was filtered off and washed several times with water giving a pale solid. The product was purified by preparative HPLC and obtained in 130 mg, 24% yield.

### 2d) Preparation of 1,3,5-tris-(N-chloroacetyl-N-methyl-4-amino-2-methyl-phenyl)-benzene (4)

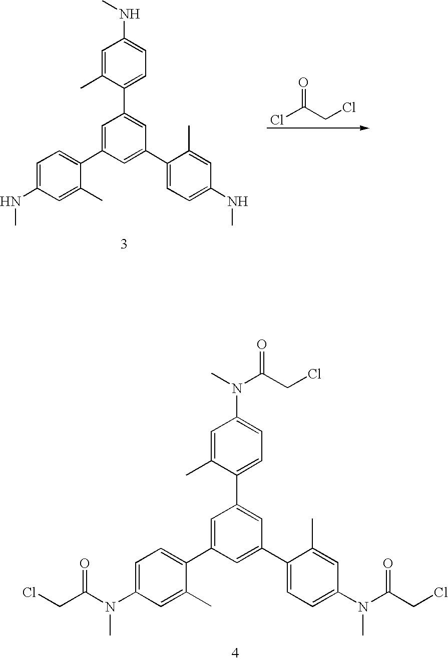

To a cooled solution (0° C.) of 3 (110 mg, 0.253 mmol) in dry DMF (5 ml) was added 2-chloroacetylchloride (Fluka, 0.07 ml, 0.884 mmol). The reaction was then stirred at room temperature for 30 min under argon. The solvent was evaporated (rotary evaporator), the product mixture dissolved in dichloromethane washed with water and dried (Na₂SO₄). The product was obtained in 109 mg, 65% yield. The structure was confirmed by LC-MS.

### 2e) Preparation of 1,3,5-tris-(N-(DO3A t-butylester-acetamido)-N-methyl-4-amino-2-methyl-phenyl)-benzene (5)

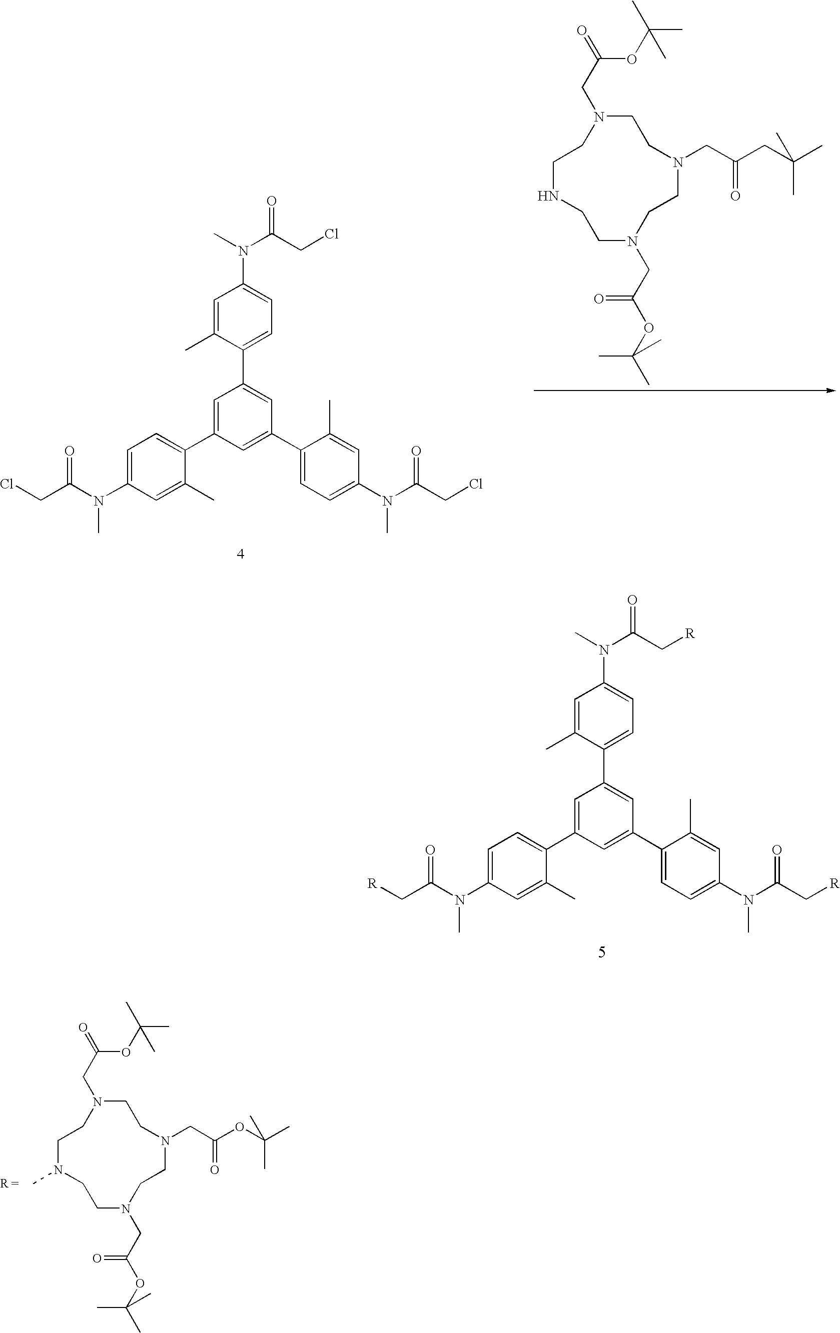

To a suspension of 4 (99 mg, 0.149 mmol) in dry acetonitrile (5 ml) were added DO3A t-butyl ester (532 mg, 0.894 mmol) and diisopropylethylamine (Fluka, 0.189 ml, 1.103 mmol). After sonication the reaction mixture was stirred at 75° C. under argon for 7 hours. The solvent was evaporated (rotary evaporator) and the crude product used in the subsequent reaction. LC-MS analysis confirmed the structure.

### 2f) Preparation of 1,3,5-tris-(N-(DO3A-acetamido)-N-methyl-4-amino-2-methyl-phenyl)-benzene (6)

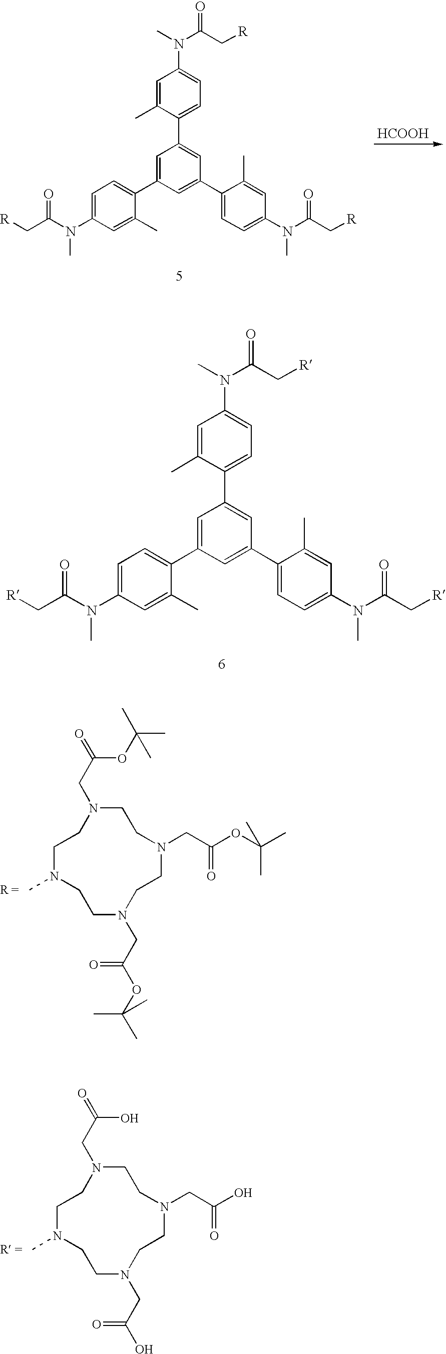

The crude product 5 was dissolved in formic acid (20 ml) and heated at reflux. The deprotection was completed after 3.5 h. The solution was evaporated (rotary evaporator). The crude product was used without further purification in the subsequent step.

### 2g) Preparation of a gadolinium derivative of 1,3,5-Tris-(N-(DO3A-acetamido)-N-methyl-4-amino-2-methyl-phenyl)-benzene (7)

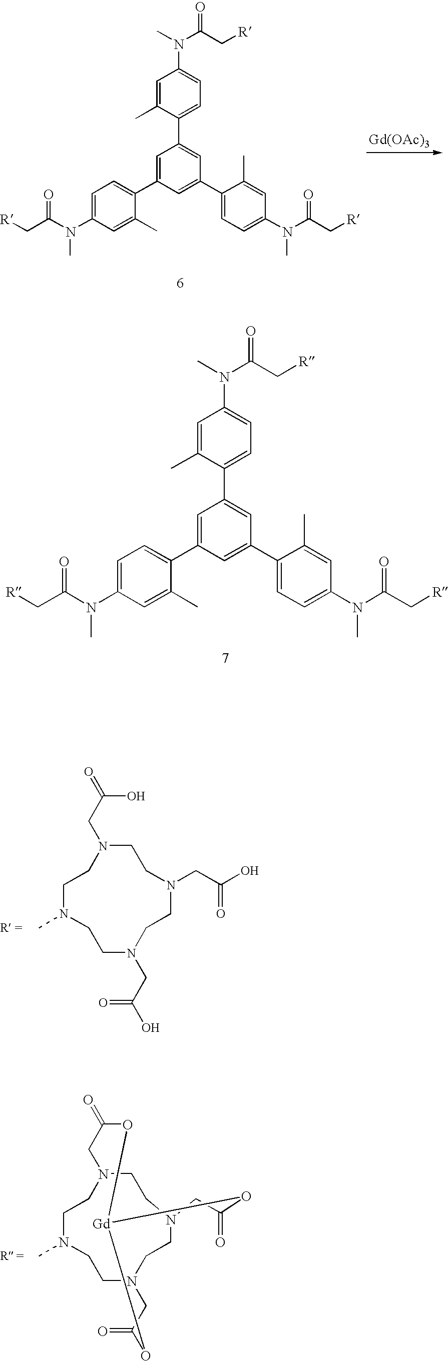

Gadolinium(III)acetate hydrate (Aldrich, 329 mg, 0.984 mmol) was added to the dissolved crude product 6 in water (15 ml). The mixture was stirred at 40° C. for 1 h. The product mixture was then purified by preparative HPLC giving 190 mg after lyophilisation, 62% yield over 3 steps. Analysis by LC MS confirmed the structure.

Compound 7 was dissolved in human blood plasma and the longitudinal relaxivity rl was measured at 37° C. at the following fields:

0.47 T, an rl of 10.6 mM¹s¹ was measured; and
1.41 T, an rl of 9.4 mM¹s¹ was measured.

Based on the above measurements an rl of 9 mM¹s¹ was calculated for a field of 3 T according to methods known in the art.

Compared to other MR contrast agent compounds known in the art, the rl at 3 T of the compound according to the invention shown above is much higher. rl values for other MRI contrast agents are at 3 T in human blood plasma at 37° C. (data published in Invest Radiol 2006, Vol. 41, 213-221):

Multihance™: rl is 6.3 mM¹s¹
Magnevist™: rl is 3.3 mM¹s¹

### Example 3

### Preparation of a gadolinium derivative of benzene-1,3,5-tris-[((DO3A-acetamido)-3-(N′-methyl-amidophenyl))-carboxamide] (13), a compound of formula (II)

### 3a) Preparation of benzene-1,3,5-tris-[(3-nitro-amidophenyl)-carboxamide] (10)

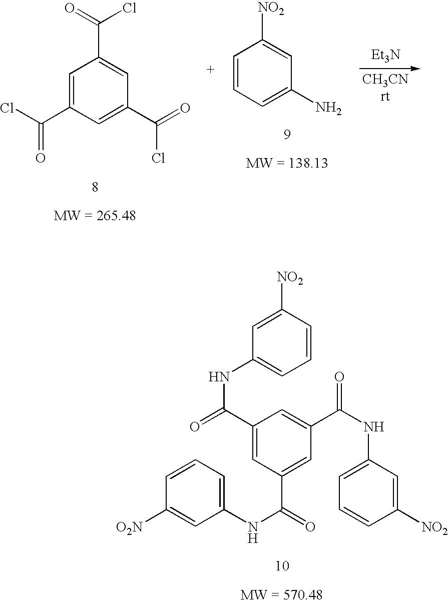

A solution of 8 (3.0 g, 11 mmol) in acetonitrile (40 ml) was added drop wise to a solution of 9 (5.2 g, 38 mmol) and Et₃N (5.2 ml, 38 mmol) in acetonitrile (90 ml) under vigorous stirring and argon atmosphere. After stirring at r t for 3 hours the solvents were removed on a rotary evaporator. The crude residue was suspended in water, filtered off and washed several times with water. The yellow precipitate was washed several times with diethyl ether to remove all residual 2 giving 10 as a pale solid (5.6 g, 87%). The structure was confirmed by LC-MS.

### 3b) Preparation of benzene-1,3,5-tris-[N-methyl-(3-nitro-amidophenyl)-carboxamide] (11)

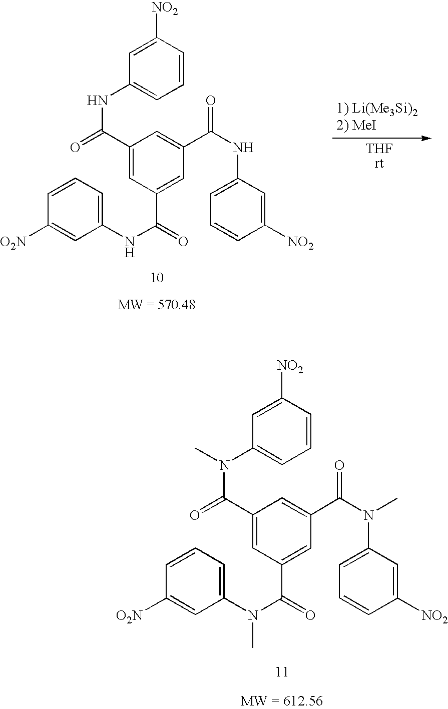

All glassware dried in oven and anhydrous THF used. Reaction performed under argon. To a suspension of 10 (5.6 g, 9.8 mmol) in THF (175 ml) Li(Me₃Si)₂ (59 ml, 59 mmol) was added under stirring. The suspension turns to solution under the formation of the anion. After 30 minutes methyl iodide (7.3 ml, 120 mmol) was added. After stirring at r t for 18 hours the solvents were removed on a rotary evaporator. The residue was suspended in water (250 ml) and neutralized with 1M HCl. The formed precipitate was filtered off and washed three times with water. The pale precipitate of 11 was dried overnight in the fume hood (5.7 g, 95%). The structure was confirmed by LC-MS.

### 3c) Preparation of benzene-1,3,5-tris-[N-methyl-(3-aniline-amidophenyl)-carboxamide] (12)

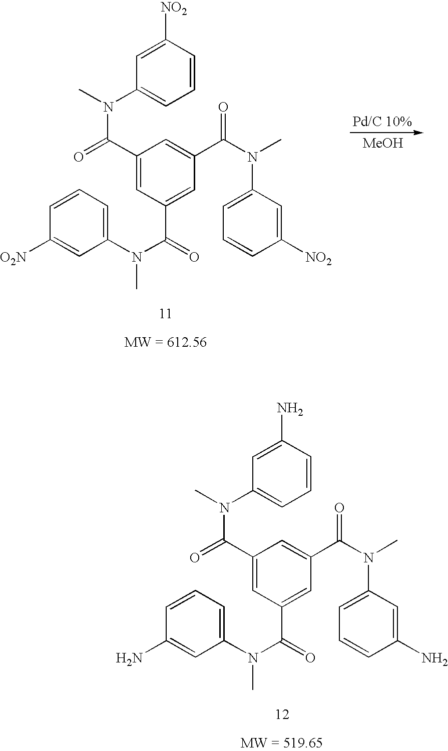

Methanol (200 ml), Pd/C (1.0 g, 10%), HCl (4.2 ml, 32%) and 11 (2.0 g, 3.3 mmol) were mixed in a 500 ml reaction flask. The mixture was hydrogenated at 60 psi on a Parr apparatus. After complete H₂ consumption, H₂O (40 ml) was added and the catalyst filtered off. The methanol was then removed on a rotary evaporator and the resulting aqueous solution was diluted to 100 ml and neutralized with solid NaHCO₃. The formed precipitate was filtered off giving 12 as a light brown solid (1.4 g, 82%). The structure was confirmed by LC-MS.

### 3d) Preparation of a gadolinium derivative of benzene-1,3,5-tris-[((DO3A-acetamido)-3-(N′-methylamido-phenyl))-carboxamide)] (13)

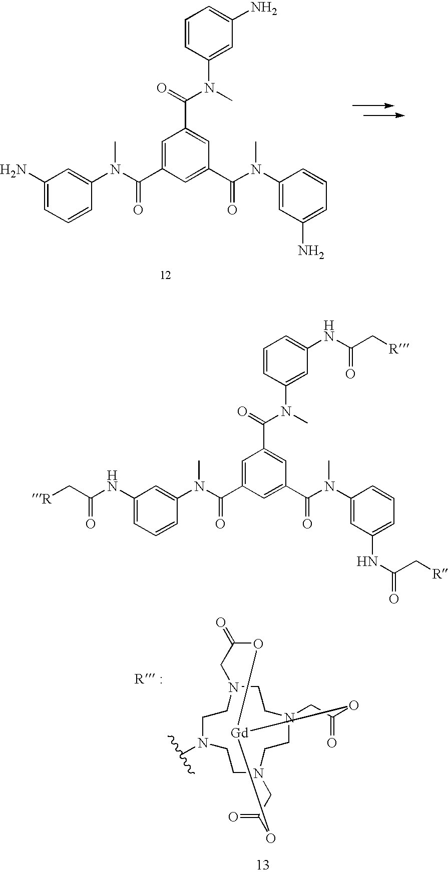

Compound 12 was transformed into the gadolinium derivative of benzene-1,3,5-tris-[((DO3A-acetamido)-3-(N′-methylamidophenyl))-carboxamide)] (13) using the same reaction conditions reported in the synthesis of the gadolinium derivative of 1,3,5-Tris-(N-(DO3A-acetamido)-N-methyl-4-amino-2-methyl-phenyl)-benzene (7), Example 2. The structure was confirmed by LC-MS.

Compound 13 was dissolved in human blood plasma and the longitudinal relaxivity rl was measured at 37° C. at the following fields:

1.5 T, an rl of 9.2 mM¹s¹ was measured; and
2.35 T, an rl of 9.5 mM¹s¹ was measured.

Based on the above measurements an rl of 9.0 mM¹s¹ was calculated for a field of 3 T according to methods known in the art.

Compared to other MR contrast agent compounds known in the art, the rl at 3 T of the compound according to the invention shown above is much higher. rl values for other MRI contrast agents are at 3 T in human blood plasma at 37° C. (data published in Invest Radiol 2006, Vol. 41, 213-221):

Multihance™: rl is 6.3 mM¹s¹
Magnevist™: rl is 3.3 mM¹s¹

### Example 4

### Preparation of 2,4,6-trimethylbenzene-1,3,5-tris-[(DO3A-(3-acet-amido-phenyl))-N-methyl-carboxamide] (35), a compound of formula (I) and a gadolinium derivative thereof (36), a compound of formula (II)

### 4a) Preparation of 1,3,5-tris-(bromomethyl)-2,4,6-trimethylbenzene (25)

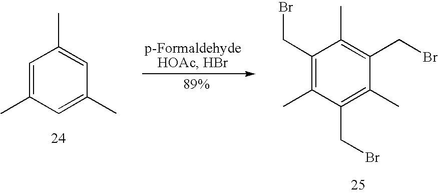

To mesitylene 24 (44.5 g, 0.37 mol) and p-formaldehyde (36.6 g, 1.22 mol) was added glacial acetic acid (185 mL) and hydrobromic acid (33% in acetic acid, 260 mL). The suspension was stirred under argon atmosphere and heated at 95° C. After 24 h the reaction mixture was crashed into water (150 mL) and stirred vigorously. The formed precipitate was filtered off and washed thoroughly with water to give compound 25 as a white powder (131.2 g, 89%).

### 4b) Preparation of 1,3,5-tris-(acetoxymethyl)-2,4,6-trimethylbenzene (26)

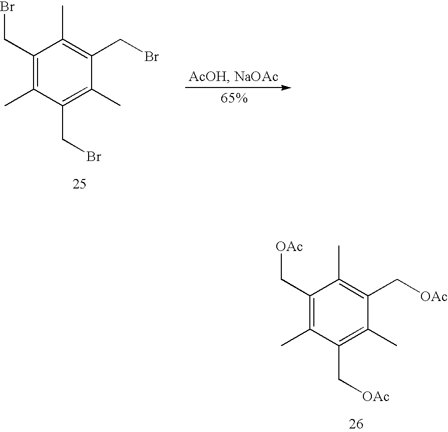

To compound 25 (15.0 g, 37.6 mmol) was added NaOAc (17.6 g, 0.21 mol) followed by glacial acetic acid (350 mL). The reaction vessel was sealed with a rubber septum that was secured with copper wire. The stirred reaction mixture was heated at 140° C. for 18 h after which the reaction mixture was concentrated to give an orange brown solid. The solid residue was suspended in water (250 mL) and extracted with dichloromethane (250 mL). The organic phase was then extracted with a saturated aqueous solution of NaHCO₃ (150 mL) followed by water (150 mL). The organic phase was then dried using Na₂SO₄ and filtered. The solvents were removed to give an orange powder which was crystallized from isopropanol. The crystals obtained were filtered off and washed with cold methanol to give triacetate 26 as a white powder (8.3 g, 65%)

### 4c) Preparation of 1,3,5-tris-(hydroxymethyl)-2,4,6-trimethylbenzene (27)

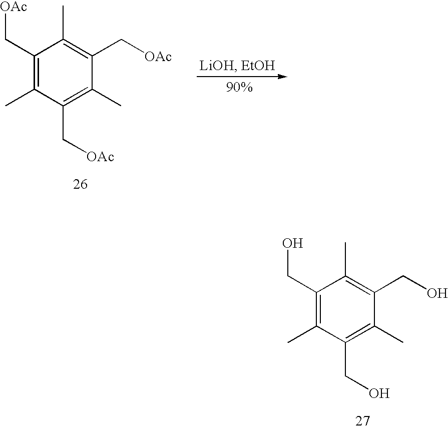

To a slurry of compound 26 (15.4 g, 46 mmol) in ethanol (310 mL) was added LiOH monohydrate (7.7 g, 184 mmol). The reaction mixture was refluxed for 18 h after which the solvents were removed. The residue was suspended in water (100 mL), filtered off and rinsed with water (200 mL) to give compound 27 as a white powder (9.0 g, 94%).

### 4d) Preparation of 2,4,6-trimethylbenzene-1,3,5-tricarboxylic acid (28)

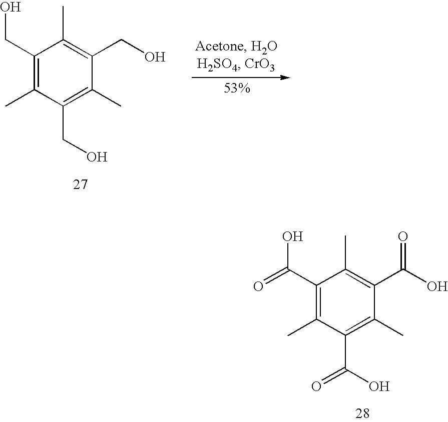

The oxidation reagent was prepared separately by portion wise addition of chromium (VI) oxide (21.4 g, 214 mmol) to a stirred solution of sulphuric acid (21.4 mL, 18 M). The now brown slurry was cooled in an ice bath and water (64 mL) was added slowly, forming a red solution. The chromium reagent was added drop wise to an ice cooled solution of compound 27 (5.0 g, 23.8 mmol) in acetone (278 mL). The reaction mixture was stirred at 0° C. for 20 min then allowed to attain room temperature during 30 minutes and then placed in an oil bath at 30° C. for 10 minutes. The reaction mixture was then crashed into water (550 mL), extracted with ether (200 mL) three times and the combined organic extracts were then washed with water (200 mL). The organic phase was dried with Na₂SO₄, filtered and evaporated to give crude 28 (4.2 g). The white crystals of crude 28 were suspended in water (70 mL) and the pH was adjusted to 7 by addition of NaOH (50 mL, 1M). The now clear solution was passed through an ion exchange column (Dowex 50×8, dimensions: D: 3 cm, L: 7 cm) end eluted with water (150 mL). The eluent was lyophilized to give a white powder that was crystallized in refluxing acetic acid (100 mL). After cooling the crystals were filtered off and rinsed with acetic acid to give compound 28 as a white powder (3.2 g, 53%).

### 4e) Preparation of 2,4,6-trimethylbenzene-1,3,5-tri-carboxylic acid chloride (29)

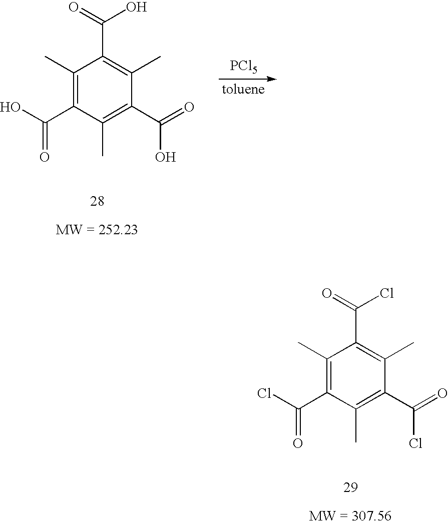

A slurry of 28 (1.0 g, 4.0 mmol) and PCl₅ (8.2 g 39.4 mmol) in toluene (10 mL) was refluxed. After 1 h, toluene and excess PCl₅ were distilled off at atmospheric pressure. A low vacuum (membrane pump) was then applied and POCl₃ was distilled off, the temperature of the oil bath never exceeding 155° C. The melt, which solidified upon cooling (Mp: 125° C.), was left to attain room temperature. The crude reaction mixture was then dissolved in Et₂O (40 mL), filtered and then concentrated to give 29 as a white powder (1.1 g, 94%).

### 4f) Preparation of 2,4,6-trimethylbenzene-1,3,5-tris-[((N-(3-nitrophenyl))carboxamide)] (31)

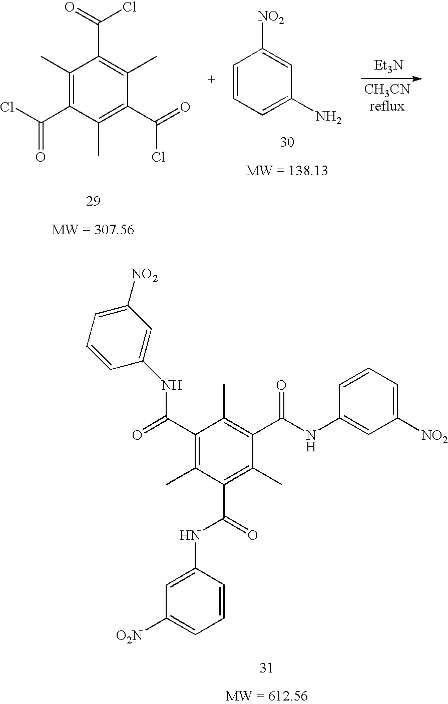

Compound 29 (0.65 g, 2.1 mmol) and nitroaniline 30 (1.0 g, 7.2 mmol) were dissolved and then refluxed in CH₃CN (15 mL) under argon atmosphere. After 3 h the reaction mixture was allowed to cool and then added drop wise to a vigorously stirred HCl (aq) solution (500 mL, 1.6 M). The formed precipitate was filtered off and rinsed with water (200 mL). The precipitate (1.3 g) was sonicated in CHCl₃/CH₃CN (50/1 mL) to give a fine suspension which was filtered off to give compound 31 (0.84 g, 65%).

### 4g) Preparation of 2,4,6-trimethylbenzene-1,3,5-tris-[(N-methyl-N-(3-nitrophenyl))-carboxamide] (32)

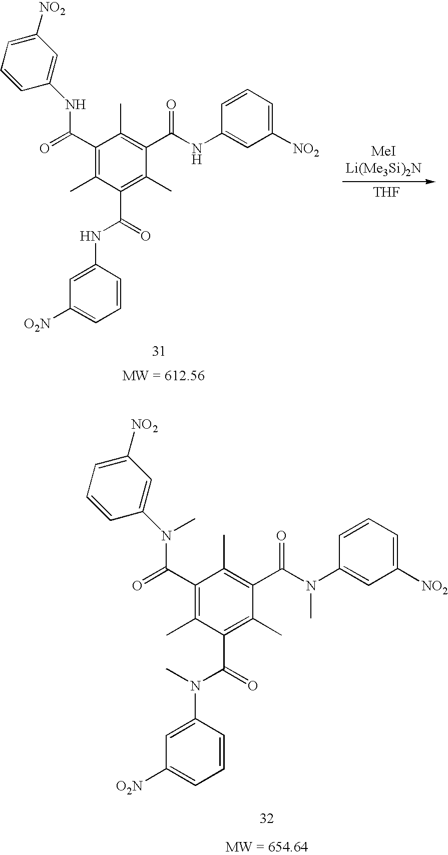

Compound 31 (0.83 g, 1.35 mmol) was dissolved in THF (40 mL) under argon atmosphere. Lithium hexamethyldisilazide (8.2 mL, 1M) was added drop wise and after 5 min MeI (1 mL, 16.1 mmol) was added. After 24 h the reaction was concentrated and then suspended in H₂O (60 mL) by sonication. The slurry was acidified by addition of HCl (2 mL, 4M) after which the fine suspension formed larger particles which were filtered off to give compound 32 as a fine olive green powder (0.79 g, 88%)

### 4h) Preparation of 2,4,6-trimethylbenzene-1,3,5-tris-[N-methyl-(3-chloroacetamido-phenyl)-carboxamide] (33)

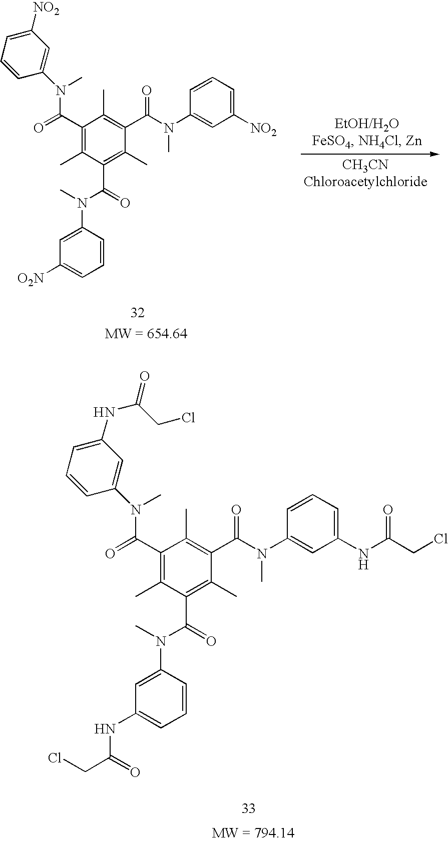

To compound 32 (1.0 g, 1.52 mmol) was added FeSO₄ heptahydrate (3.8 g, 13.7 mmol), NH₄Cl (2.0 g, 25.5 mmol) and ethanol/water (60 mL, 4/1 ratio). The formed slurry was stirred at 80° C. and zinc powder (0.9 g, 13.7 mmol) was added. After 2 h the reaction was allowed to cool and the slurry was filtered. The filtrate was concentrated and sonicated in acetonitrile (100 mL) to form a slurry which was filtered. To the filtrate was added chloroacetyl chloride (0.73 mL, 9.1 mmol) after which a slurry was formed. After 30 min the reaction mixture was filtered and the precipitate, containing a mixture of mono, bis- and tris-acetylated aniline, was dissolved in DMA (dimethylacetamide, 25 mL). To the solution was added chloroacetyl chloride (1 mL, 12.5 mmol) and triethylamine (1 mL, 7.2 mmol). After 30 min the two solutions (acetonitrile and DMA) were combined and crashed into water (750 mL). The formed precipitate was filtered off and washed with additional water to give compound 33 (0.82 g, 68%).

### 4i) Preparation of 2,4,6-trimethylbenzene-1,3,5-tris-[((DO3A-t-butylester)-(3-acet-amidophenyl))-N-methyl-carboxamide] (34)

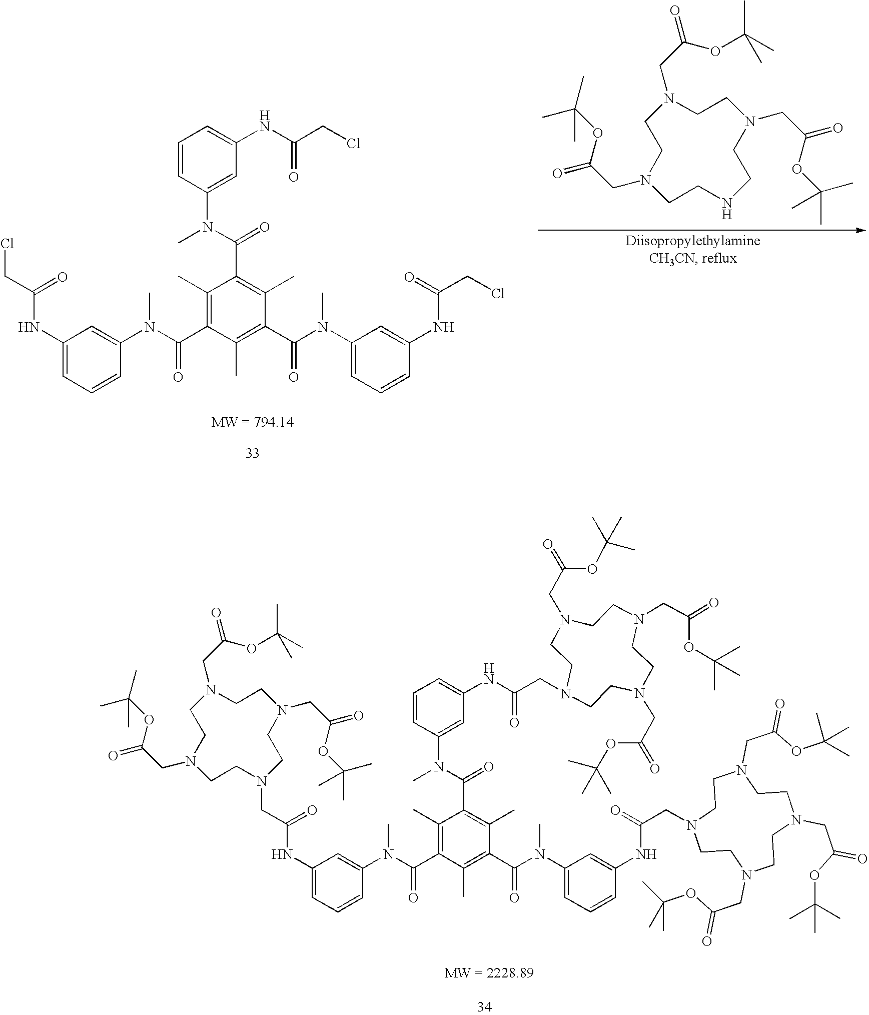

Compound 33 (0.81 g, 1.0 mmol) and DO3A(t-Bu)₃ (2.1 g, 4.1 mmol) were dissolved in CH₃CN (30 mL) and N,N-diisopropylethylamine (1.2 mL, 7.3 mmol) was added under argon atmosphere. The reaction mixture was refluxed for 19 h after which the solvents were removed to give 34 as a brown syrup which was used without purification in the next step.

### 4j) Preparation of 2,4,6-trimethylbenzene-1,3,5-tris-[(DO3A-(3-acetamidophenyl))-N-methyl-carboxamide] (35)

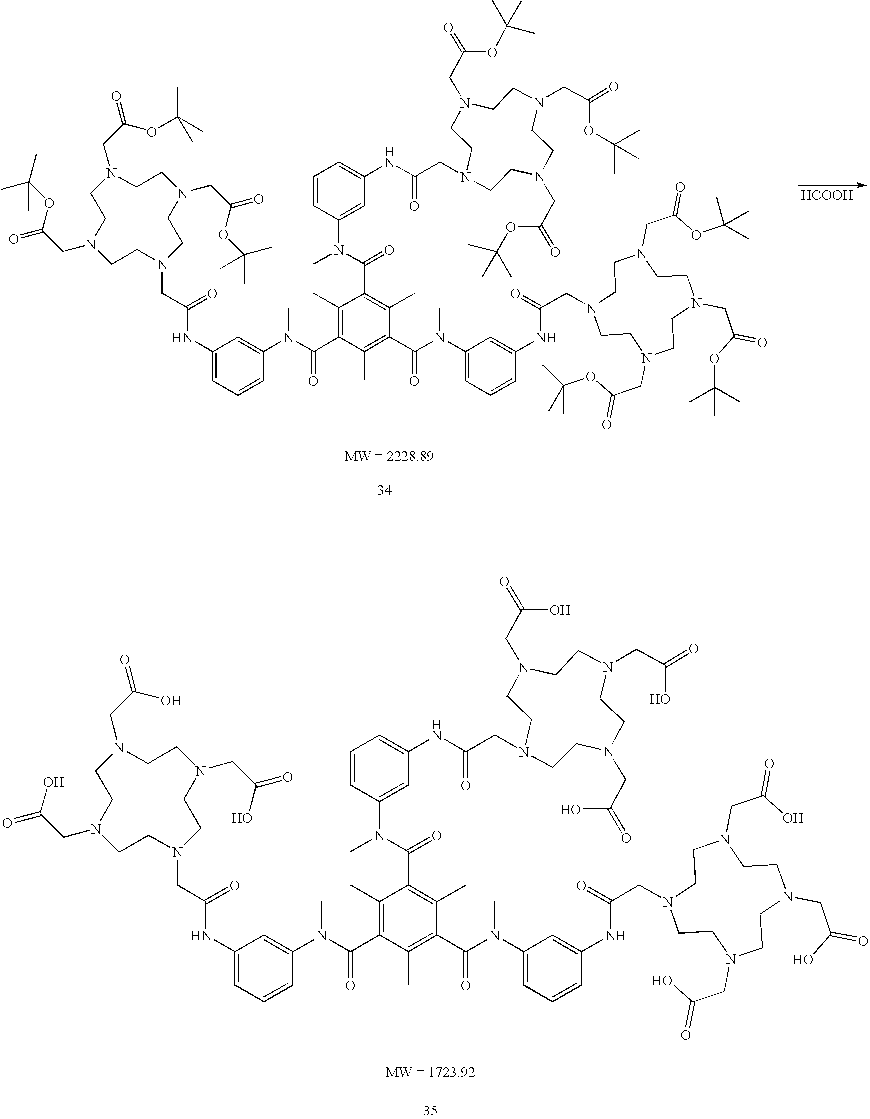

Compound 34 (crude reaction mixture originating from 0.81 g 33) was dissolved in formic acid (25 mL) and refluxed for 1 h after which the solvent was removed to give compound 35 as a brown syrup which was used in the next step without purification.

### 4k) Preparation of the gadolinium derivative of 2,4,6-trimethylbenzene-1,3,5-tris-[(DO3A-(3-acetamidophenyl))-N-methyl-carboxamide] (36)

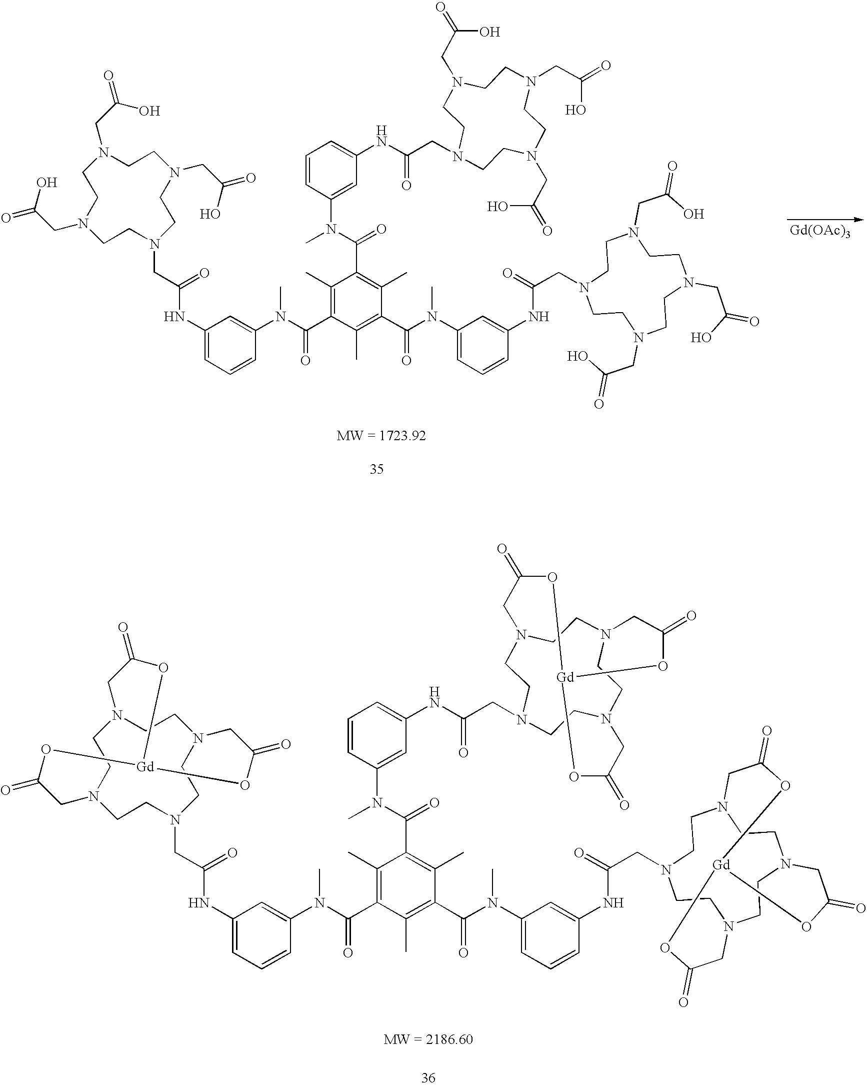

The crude reaction mixture containing 35 (originating from 0.81 g 33) was dissolved in H₂O (25 mL) and Gd(OAc)₃ (2.0 g, 6.0 mmol) was added to the stirred reaction mixture at room temperature. KOAc was added to adjust the pH to 5 and vacuum was periodically applied to remove formed acetic acid, additional H₂O was added to maintain the reaction volume. After 24 h the reaction was concentrated and preparative HPLC gave compound 36 as an off white powder (0.9 g, 41% over three steps).

Compound 36 was dissolved in human blood plasma and the longitudinal relaxivity rl was measured at 37° C. at the following fields:

0.235 T, an rl of 9.3 mM¹s¹ measured; and
0.47 T, an rl of 8.8 mM¹s¹ was measured; and
1.41 T, an rl of 7.3 mM¹s¹ was measured.

Based on the above measurements an rl of 8.5 mM¹s¹ was calculated for a field of 3 T according to methods known in the art.

Compared to other MR contrast agent compounds known in the art, the rl at 3 T of the compound according to the invention shown above is much higher. rl values for other MRI contrast agents are at 3 T in human blood plasma at 37° C. (data published in Invest Radiol 2006, Vol. 41, 213-221):

Multihance™: rl is 6.3 mM¹s¹
Magnevist™: rl is 3.3 mM¹s¹

### Example 5

### Preparation of 2,4,6-trimethylbenzene-1,3,5-tris-[(2-DO3A-propyl)-3-aminophenyl]-carboxamide] (39), a compound of formula (I) and a gadolinium derivative thereof (40), a compound of formula (II)

### 5a) Preparation of 2,4,6-Trimethylbenzene-1,3,5-tris-[N-methyl-((2-chloropropyl)-3-amidophenyl)-carboxamide)] (37)

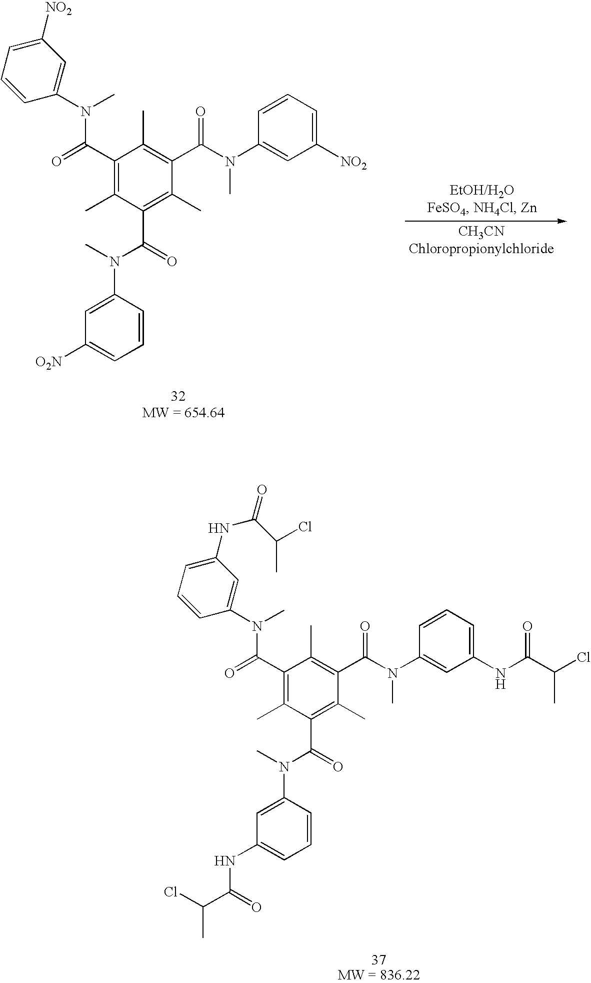

To compound 32 (0.28 g, 0.43 mmol) was added FeSO₄ heptahydrate (1.1 g, 4.0 mmol), NH₄Cl (0.5 g, 9.3 mmol) and ethanol/water (20 mL, 4/1 ratio). The formed slurry was stirred at 80° C. and zinc powder (0.25 g, 3.8 mmol) was added. After 1.5 h the reaction was allowed to cool and the slurry was filtered. The filtrate was concentrated and sonicated in acetonitrile (20 mL) to form a slurry which was filtered and diluted with additional acetonitrile (20 mL). 2-Propionylchloride (0.42 mL, 4.3 mmol) was added after which a slurry was formed. Triethylamine (0.5 mL, 3.6 mmol) was added. After 30 min the reaction mixture was concentrated and crashed into water (75 mL). The formed precipitate was sonicated, filtered off, and washed with additional water to give compound 37 (0.25 g, 70%).

### 5b) Preparation of 2,4,6-trimethylbenzene-1,3,5-tris-[N-methyl-((2-(DO3A-t-butyl-ester)-propyl)-3-amidophenyl)-carboxamide)] (38)

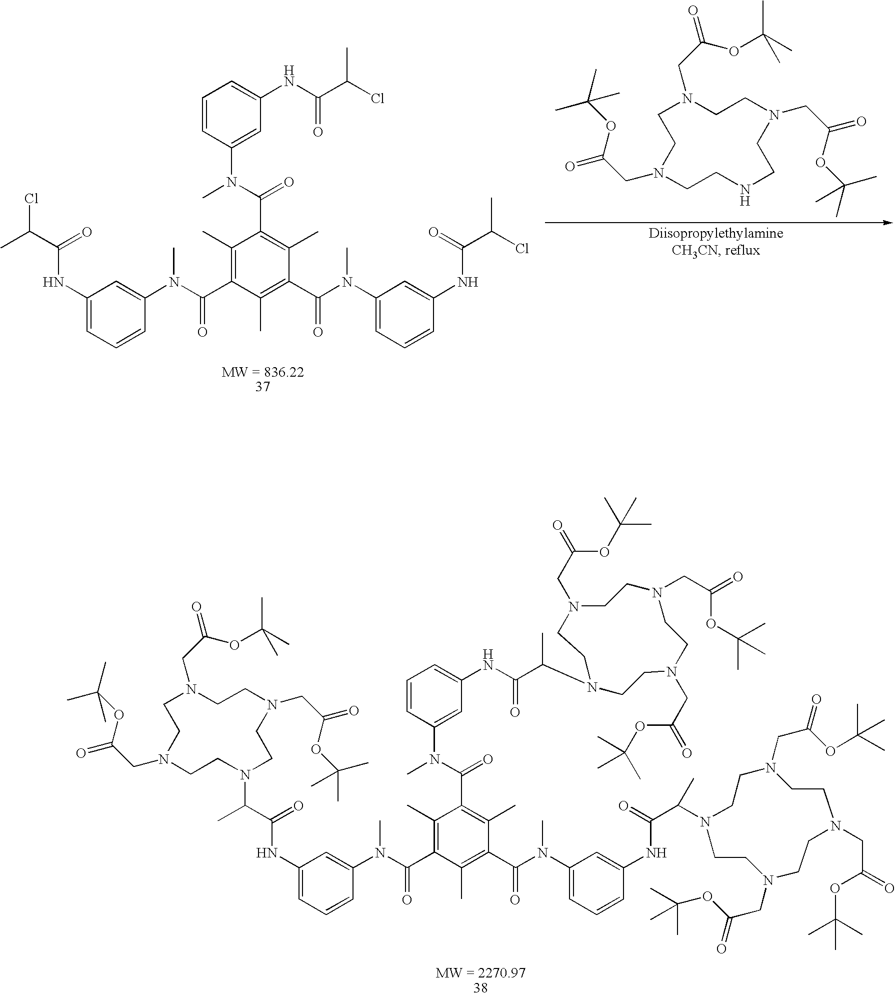

Compound 37 (0.21 g, 0.25 mmol) and DO3A(t-Bu)₃ (0.52 g, 1.0 mmol) were dissolved in CH₃CN (8 mL) and N,N-Diisopropylethylamine (0.3 mL, 1.8 mmol) was added under argon atmosphere. The reaction mixture was refluxed for 72 h after which the solvents were removed to give 38 as a brown syrup which was used without purification in the next step.

### 5c) Preparation of 2,4,6-trimethylbenzene-1,3,5-tris-[N-methyl-((2-DO3A-propyl)-3-amidophenyl)-carboxamide)](39)

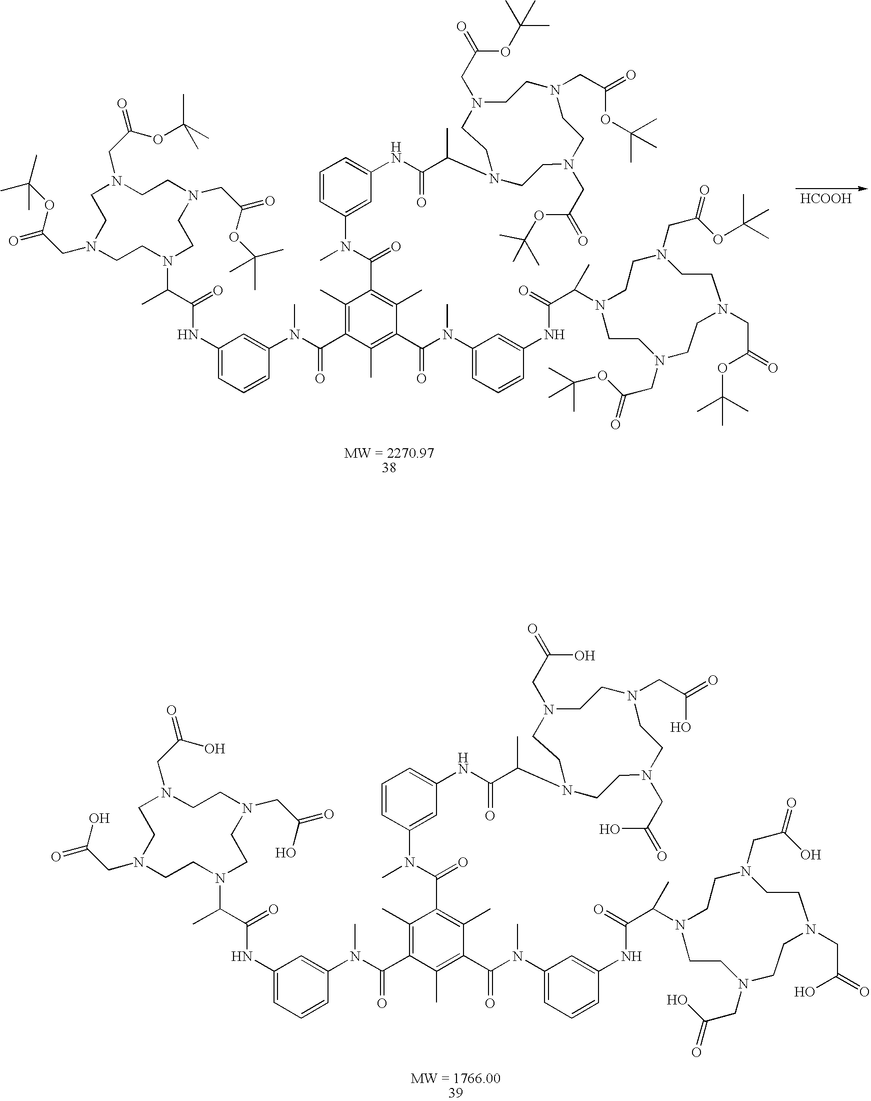

Compound 38 (crude reaction mixture originating from 0.21 g 37) was dissolved in formic acid (8 mL) and refluxed for 1 h after which the solvent was removed to give compound 39 as a brown syrup which was used in the next step without purification.

### 5d) Preparation of a gadolinium derivative of 2,4,6-trimethylbenzene-1,3,5-tris-[N-methyl-((2-DO3A-propyl)-3-amidophenyl)-carboxamide)] (40)

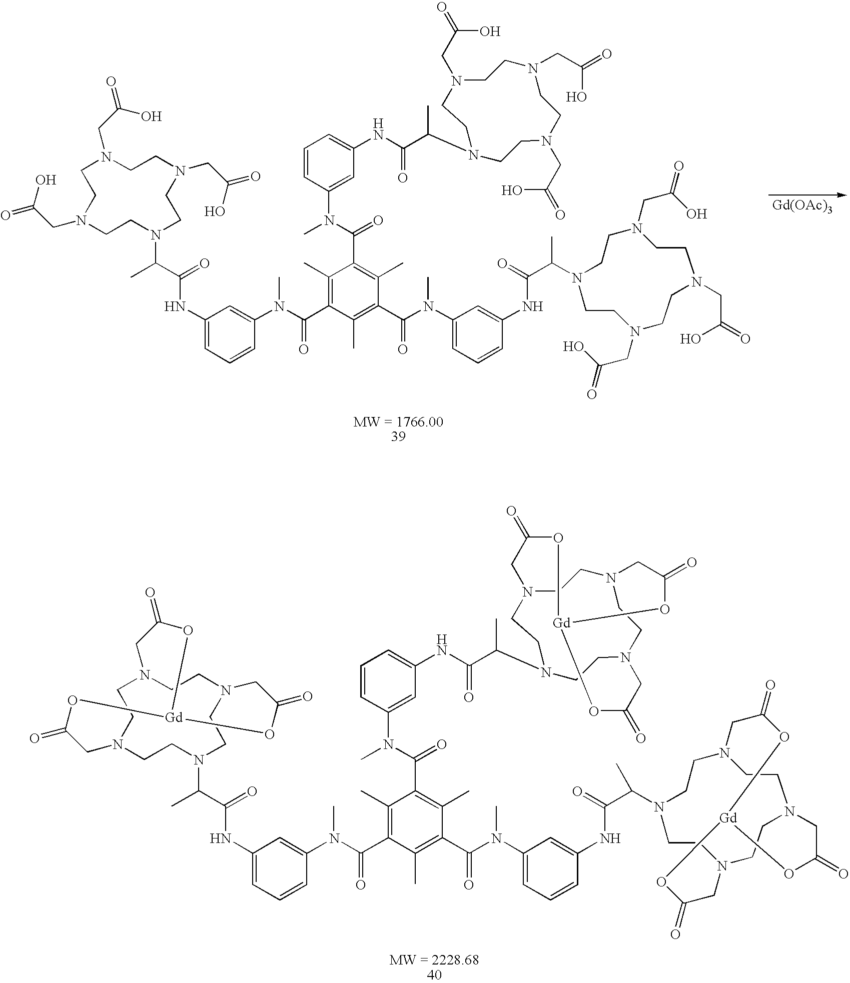

The crude reaction mixture containing 39 (originating from 0.21 g 37) was dissolved in H₂O (10 mL) and Gd(OAc)₃ (0.5 g, 1.5 mmol) was added to the stirred reaction mixture at room temperature. KOAc was added to adjust the pH to 5. After 24 h the reaction mixture was concentrated and preparative HPLC gave compound 40 as a white powder. This batch was combined with a second batch to give (0.43 g, 17% over three steps originating from 0.96 g 37.

Compound 40 was dissolved in human blood plasma and the longitudinal relaxivity rl was measured at 37° C. at the following fields:

0.235 T, an rl of 10.1 mM¹s¹ measured; and
0.47 T, an rl of 8.6 mM¹s¹ was measured; and
1.41 T, an rl of 9.1 mM¹s¹ was measured.

Based on the above measurements an rl of 9 mM¹s¹ was calculated for a field of 3 T according to methods known in the art.

Compared to other MR contrast agent compounds known in the art, the rl at 3 T of the compound according to the invention shown above is much higher. rl values for other MRI contrast agents are at 3 T in human blood plasma at 37° C. (data published in Invest Radiol 2006, Vol. 41, 213-221):

Multihance™: rl is 6.3 mM¹s¹
Magnevist™: rl is 3.3 mM¹s¹

### Example 6

### Preparation of 2,4,6-trimethylbenzene-1,3,5-tris-[N-methyl-[(DO3A-(3-acetamidophenyl))-N-methyl-carboxamide] (42), a compound of formula (I) and a gadolinium derivative thereof (43), a compound of formula (II)

### 6a) Preparation of 2,4,6-trimethylbenzene-1,3,5-tris-[N-methyl-((2-(DO3A-t-butyl-ester)-propyl)-3-(N′-methyl-amidophenyl))-carboxamide)] (41)

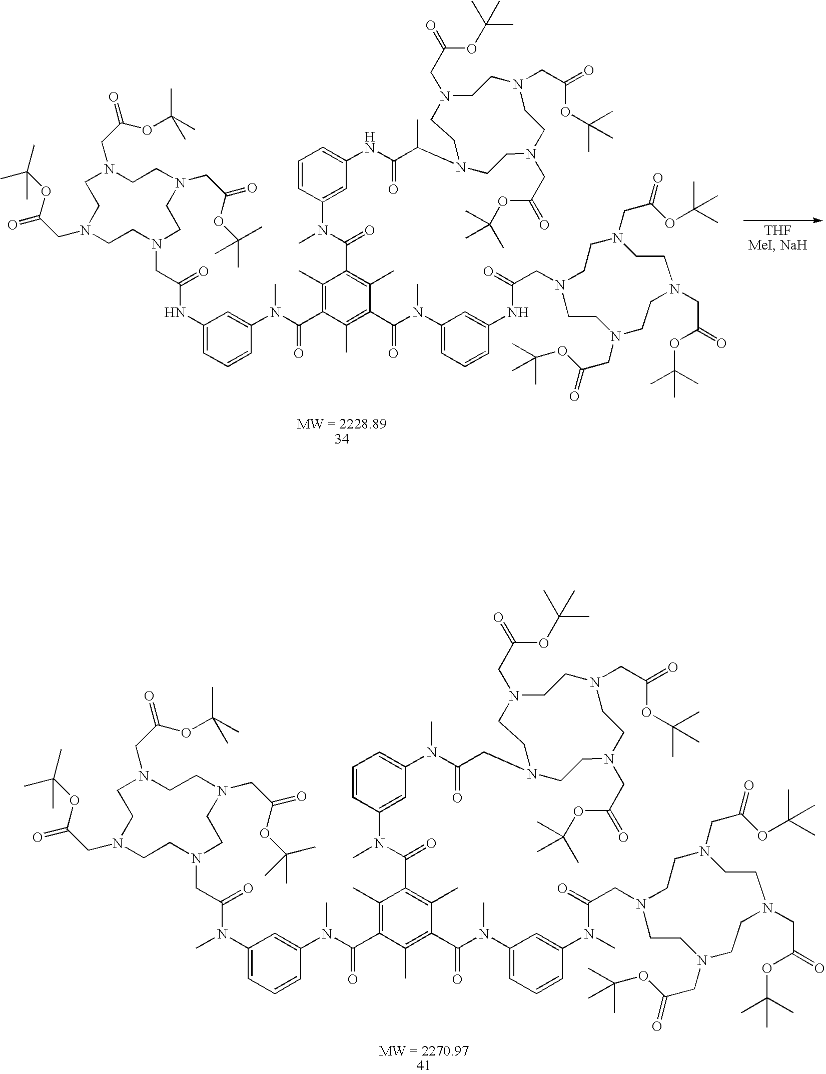

The crude compound 34 (originating from 0.50 g 33) was suspended in THF (20 mL) by sonication. MeI (0.5 mL, 7.5 mmol) was added followed by NaH (60% in mineral oil, 0.15 g, 3.8 mmol). After 30 min the reaction was diluted with THF (100 mL) and additional MeI (0.5 mL, 7.5 mmol) and NaH (60%, 0.15 g, 7.5 mmol) were added. After 1 h additional NaH (60%, 0.15 g, 7.5 mmol) was added. After 2 h the reaction mixture was concentrated to give a brown foam to which was added an aqueous solution of HCOOH (0.1%, 100 mL). Mechanical grinding and sonication gave a fine slurry which was diluted with CH₂Cl₂ (100 mL) and extracted. The organic phase was then washed with water (100 mL) and then dried with MgSO₄, filtered and concentrated to give 1.6 g of 41, a brownish fine powder which was used without further purification in the next step.

### 6b) Preparation of 2,4,6-trimethylbenzene-1,3,5-tris-[N-methyl-((2-DO3A-propyl)-3-(N′-methyl-amidophenyl))-carboxamide)] (42)

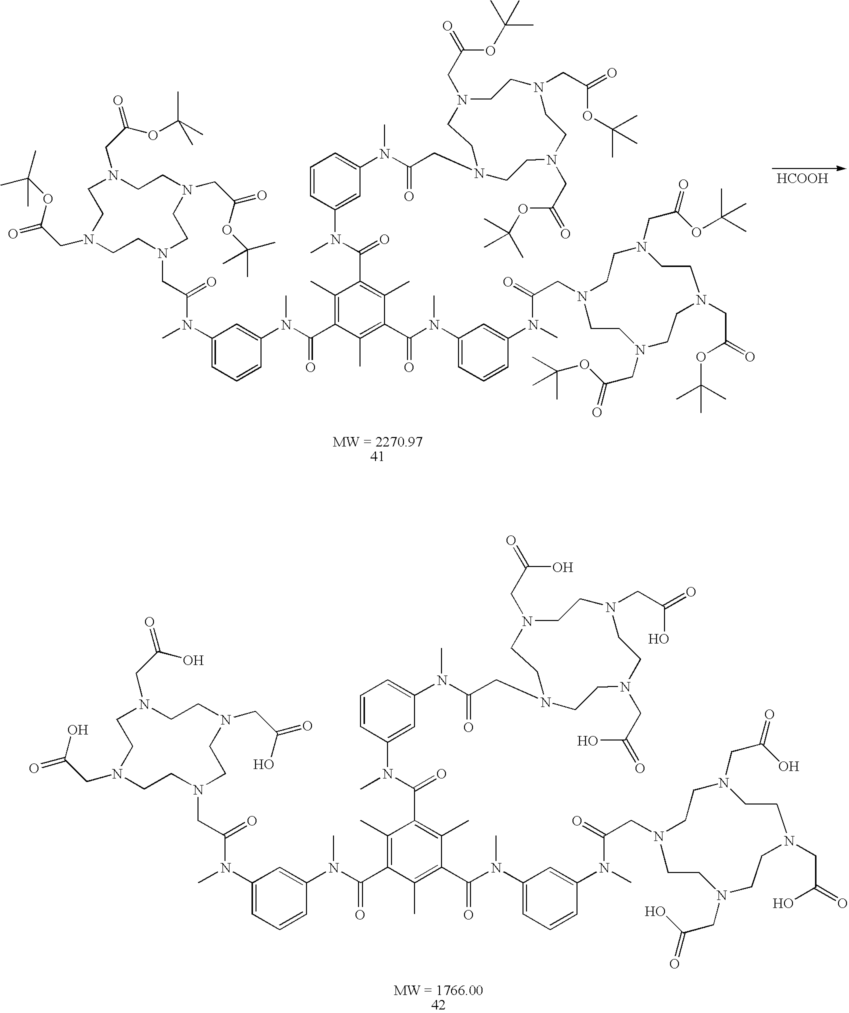

Compound 41 (crude reaction mixture originating from 0.50 g 37) was dissolved in formic acid (20 mL) and refluxed for 90 min after which the solvent was removed to give compound 42 as a brown syrup which was used in the next step without purification.

### 6c) Preparation of a gadolinium derivative of 2,4,6-trimethylbenzene-1,3,5-tris-[N-methyl-((2-DO3A-propyl)-3-(N′-methyl-amidophenyl))-carboxamide)] (43)

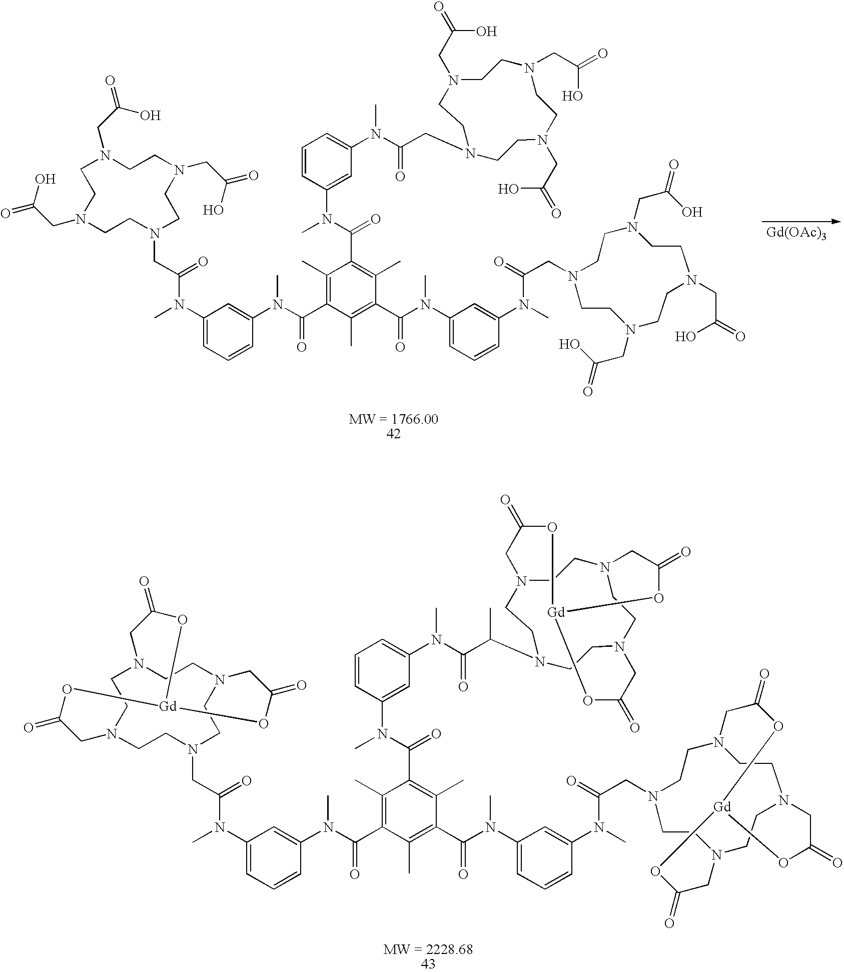

The crude reaction mixture containing 42 (originating from 0.50 g 37) was dissolved in H₂O (20 mL) and Gd(OAc)₃ (1.3 g, 3.9 mmol) was at room temperature. KOAc was added to adjust the pH to 4. After 2 h the reaction was washed with CH₂Cl₂ (30 mL) and the water phase was filtered (PALL, 0.45μ PTFE ACRODISC CR) to give a clear brownish solution. The solution was concentrated and preparative HPLC gave compound 43 as an off white powder (0.53 g, 38% over four steps).

Compound 43 was dissolved in human blood plasma and the longitudinal relaxivity rl was measured at 37° C. at the following fields:

0.235 T, an rl of 12 mM¹s¹ measured; and
0.47 T, an rl of 10.6 mM¹s¹ was measured; and
1.41 T, an rl of 9 mM¹s¹ was measured.

Based on the above measurements an rl of 8.2 mM¹s¹ was calculated for a field of 3 T according to methods known in the art.

Compared to other MR contrast agent compounds known in the art, the rl at 3 T of the compound according to the invention shown above is much higher. rl values for other MRI contrast agents are at 3 T in human blood plasma at 37° C. (data published in Invest Radiol 2006, Vol. 41, 213-221):

Multihance™: rl is 6.3 mM¹s¹
Magnevist™: rl is 3.3 mM¹s¹

### Example 7

### Preparation of 2,4,6-trimethylbenzene-1,3,5-tris-[N-methyl-((2-DO3A-propyl)-3-(N′-methyl-amidophenyl))-carboxamide)] (45), a compound of formula (I) and a gadolinium derivative thereof (46), a compound of formula (II)

### 7a) Preparation of 2,4,6-trimethylbenzene-1,3,5-tris-[N-methyl-((2-(DO3A-t-butyl-ester)-propyl)-3-(N′-methyl-amidophenyl))-carboxamide)] (44)

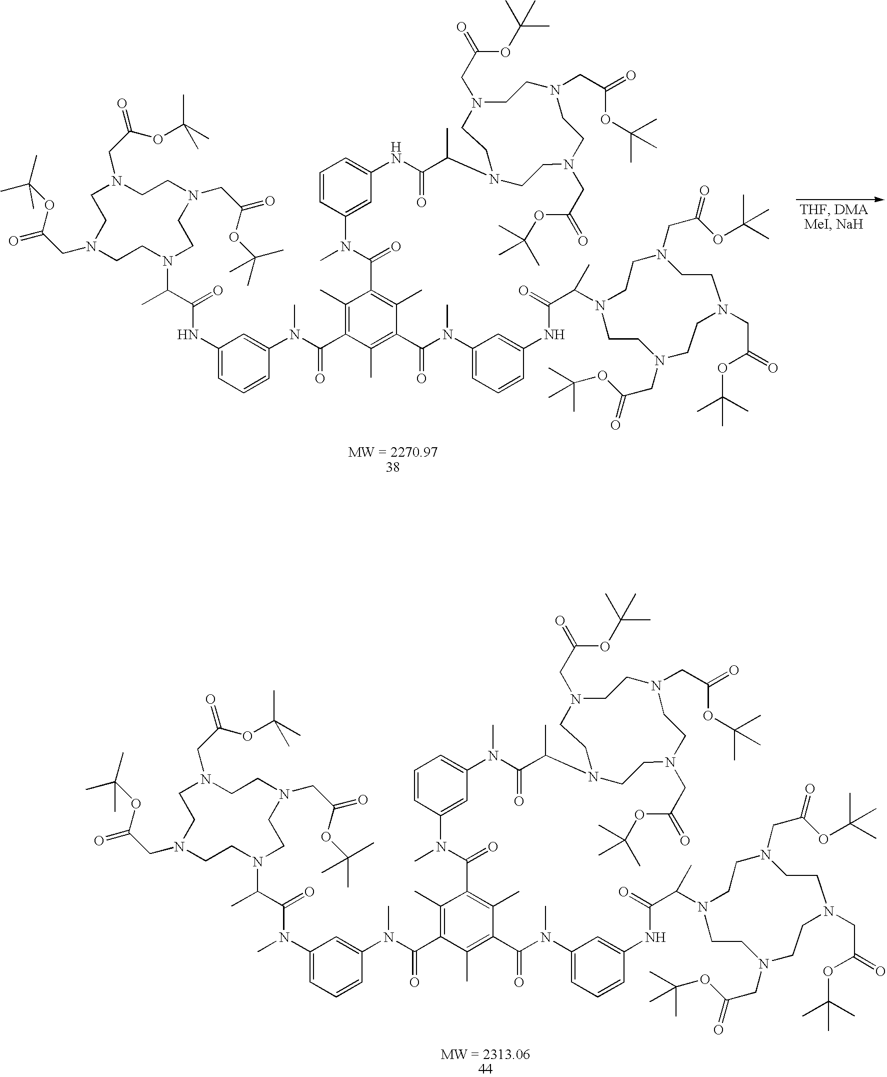

To compound 38 (crude reaction mixture originating from 0.45 g 37) was added THF (70 mL) and DMA (5 mL), sonication gave a yellowish solution with a precipitate. The reaction mixture was stirred under argon atmosphere and MeI (0.4 mL, 6.4 mmol) was added followed by NaH (60%, 0.13 g, 3.3 mmol). After 120 min additional MeI (0.4 mL, 6.4 mmol) and NaH (60%, 0.13 g, 3.3 mmol) was added. After 150 min additional NaH (60%, 0.13 g, 3.3 mmol) was added After 200 min the reaction mixture was concentrated and dissolved in dichloromethane (200 mL). The organic phase was extracted with HCOOH (0.5%, 200 mL) followed by water (200 mL), dried with MgSO₄, filtered and concentrated. The crude reaction mixture was used without further purification in the next step.

### 7b) Preparation of 2,4,6-trimethylbenzene-1,3,5-tris-[N-ethyl((2-DO3A-propyl)-3-(N′-methyl-amidophenyl))-carboxamide)] (45)

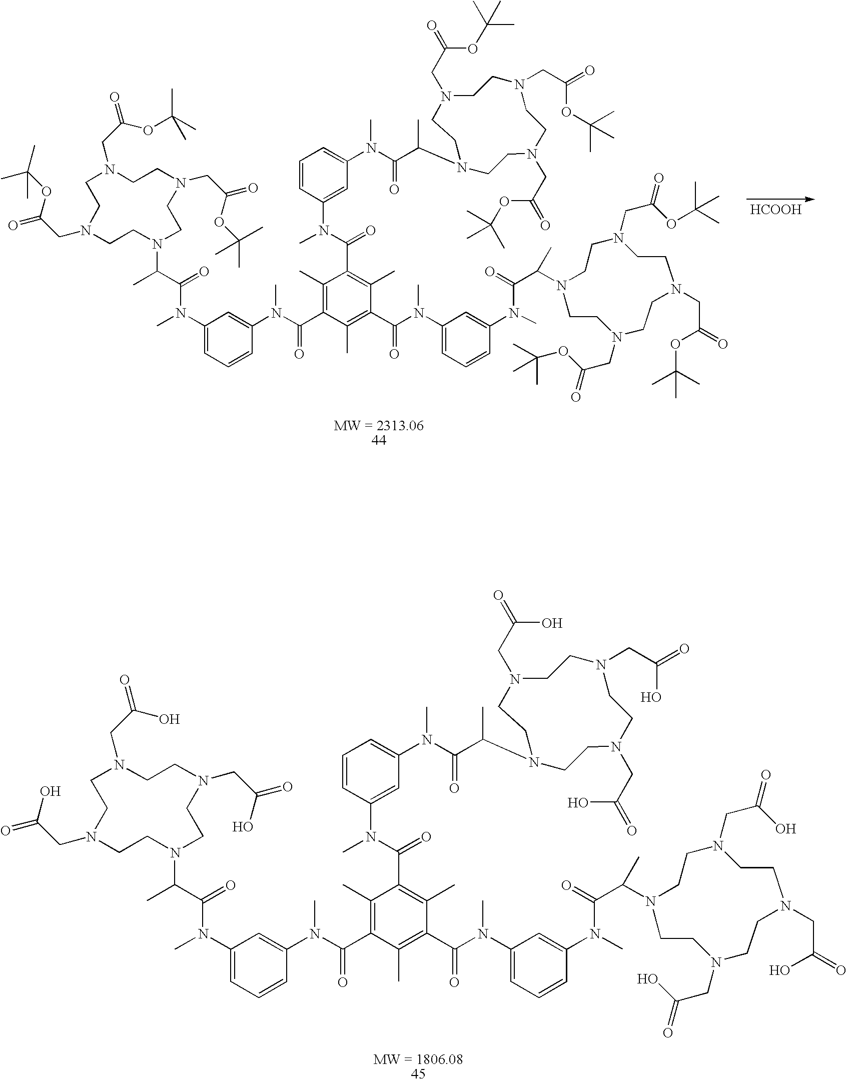

To compound 44 (crude reaction mixture originating from 0.45 g 37) dissolved in dichloromethane (25 mL) was added TFA (10 mL) under argon atmosphere. The reaction mixture was refluxed for 3 h after which the solvents were removed to give compound 45 as a brown syrup which was used in the next step without purification.

### 7c) Preparation of a gadolinium derivative of 2,4,6-trimethylbenzene-1,3,5-tris-[N-methyl-((2-DO3A-propyl)-3-(N′-methyl-amidophenyl))-carboxamide)] (46)

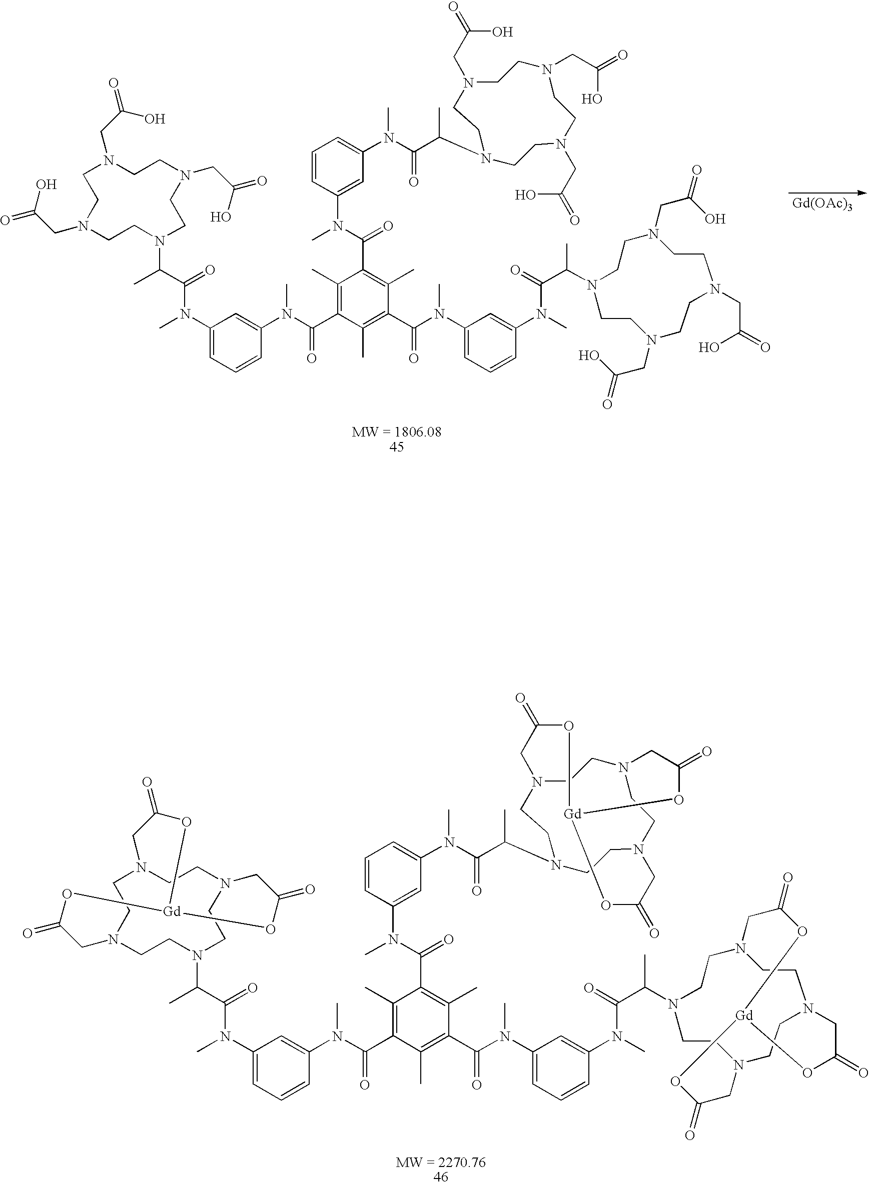

The crude reaction mixture containing 45 (originating from 0.45 g 37) was dissolved in H₂O (40 mL) and Gd(OAc)₃ (1.0 g, 3.0 mmol) was added to the stirred reaction mixture at room temperature. KOAc was added to adjust the pH to 5. After 24 h the reaction was concentrated and preparative HPLC gave compound 46 as a white powder (0.17 g, 14% over four steps).

Compound 46 was dissolved in human blood plasma and the longitudinal relaxivity rl was measured at 37° C. at the following fields:

0.235 T, an rl of 10.9 mM¹s¹ measured; and
0.47 T, an rl of 10.1 mM¹s¹ was measured; and
1.41 T, an rl of 8.9 mM¹s¹ was measured.

Based on the above measurements an rl of 9 mM¹s¹ was calculated for a field of 3 T according to methods known in the art.

Compared to other MR contrast agent compounds known in the art, the rl at 3 T of the compound according to the invention shown above is much higher. rl values for other MRI contrast agents are at 3 T in human blood plasma at 37° C. (data published in Invest Radiol 2006, Vol. 41, 213-221):

Multihance™: rl is 6.3 mM¹s¹
Magnevist™: rl is 3.3 mM¹s¹

### Example 8

### Preparation of 2,7,12-tris-(DOTA-amido)-5,5′,10,10′,15,15′-hexakis-methoxymethyl)-truxene (51), a compound of formula (I) and a gadolinium derivative thereof (52), a compound of formula (II)

### 8a) Preparation of 5,5′,10,10′,15,15′-hexakis(methoxymethyl)truxene (48)

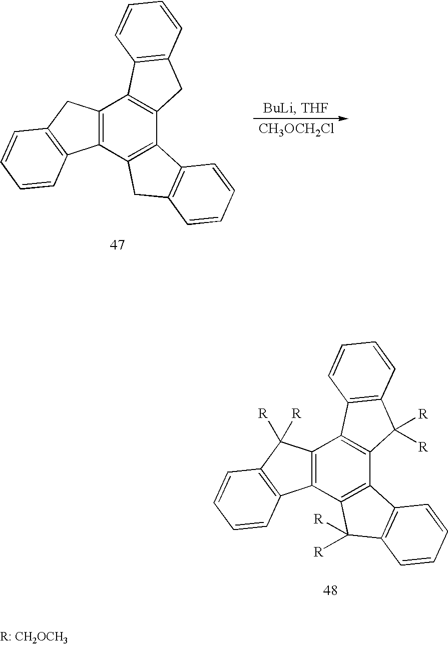

Compound 47 (5 g, 14.6 mmol) is dissolved in THF (100 mL) and then cooled to −70° C. under nitrogen atmosphere. n-butyl lithium (22 mL, 2M in cyclohexane) is then added and the reaction mixture is allowed to reach ambient temperature. Then chloromethyl methyl ether (3.66 mL, 48.2 mmol) is added and the reaction is stirred at ambient temperature for 2 h. The reaction mixture is then extracted with brine and dichloromethane. The organic phase is dried and concentrated. The residue is dissolved in THF (100 mL) and then cooled to −70° C. under nitrogen atmosphere. n-butyl lithium (22 mL, 2M in cyclohexane) is then added and the reaction mixture is allowed to reach ambient temperature. Then chloromethyl methyl ether (3.66 mL, 48.2 mmol) is added and the reaction is stirred at ambient temperature for 2 h. The reaction mixture is then extracted with brine and dichloromethane. The organic phase is dried and concentrated to give compound 48 (8.8 g, 14.6 mmol).

### 8b) Preparation of 2,7,12-trinitro-5,5′,10,10′15,15′-hexakis(methoxymethyl)-truxene (49)

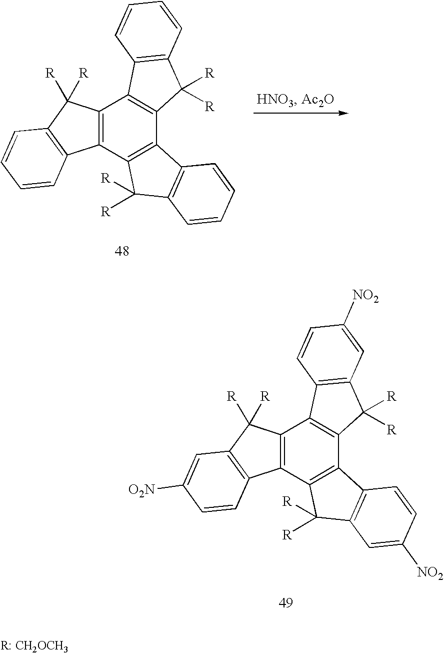

Compound 48 (8.8 g, 14.6 mmol) is slowly added to an ice cooled mixture of fuming HNO₃ (50 mL) and acetic anhydride (8.3 mL, 88 mmol). It is taken care that the temperature never exceeds 5° C. The reaction mixture is then poured into ice-water and the precipitate is filtered off to give compound 49.

### 8c) Preparation of 2,7,12-triamino-5,5′,10,10′,15,15′-hexakis(methoxymethyl)-truxene (50)

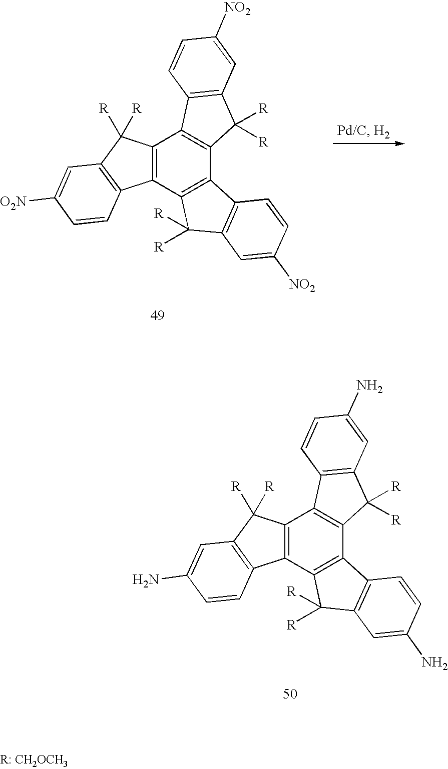

Compound 49 (10.8 g, 14.6 mmol) is dissolved in THF (100 mL) and Pd/C (3 g, 10%) is added. The reaction mixture is subjected to molecular hydrogen at 10 bar in a high pressure reactor under vigorous stirring. After 3 h the reaction mixture is filtered and concentrated to give compound 50.

### 8d) Preparation of 2,7,12-tris-(DOTA-amido)-5,5′,10,10′,15,15′-hexakis(methoxy-methyl)truxene (51)

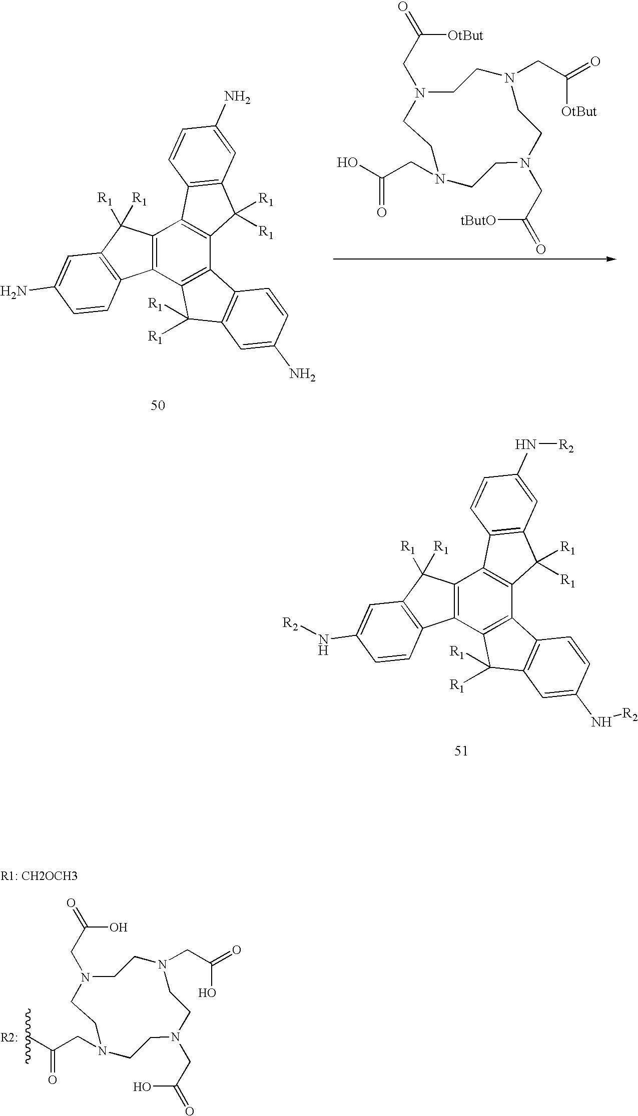

4,7,10-Tricarboxymethyl-tert-butyl ester 1,4,7,10-tetraazacyclododecane-1-acetate (DOTA(tBu)₃) (33.7 g, 48.2 mmol), which is obtained as described in Heppeler, A; Chem. Eur. J. 1999, 5, 1974-1981, HATU (O-(7-azabenzotriazole-1-yl)-1,1,3,3-tetramethyluronium hexafluorophosphate) (18.3 mL, 48.2 mmol), and DIPEA ((N,N′-diisopropylethylamine) (8.2 mL, 48.2 mmol) are preincubated in DMF (500 mL). After 10 min, compound 50 (9.5 g, 14.6 mmol) and DIPEA (8.1 mL, 48.2 mmol) dissolved in DMF (100 mL) is added. The reaction mixture is stirred for 3 h and then diluted with water and extracted with ethyl acetate. The organic phase is dried and concentrated and dissolved in formic acid (100 mL). The solution obtained is refluxed for 1 h and then concentrated to give compound 51.

### 8e) Preparation of a gadolinium derivative of 2,7,12-tris(DOTA-amido)-5,5,10,10′,15,15′-hexakis(methoxymethyl)truxene (52)

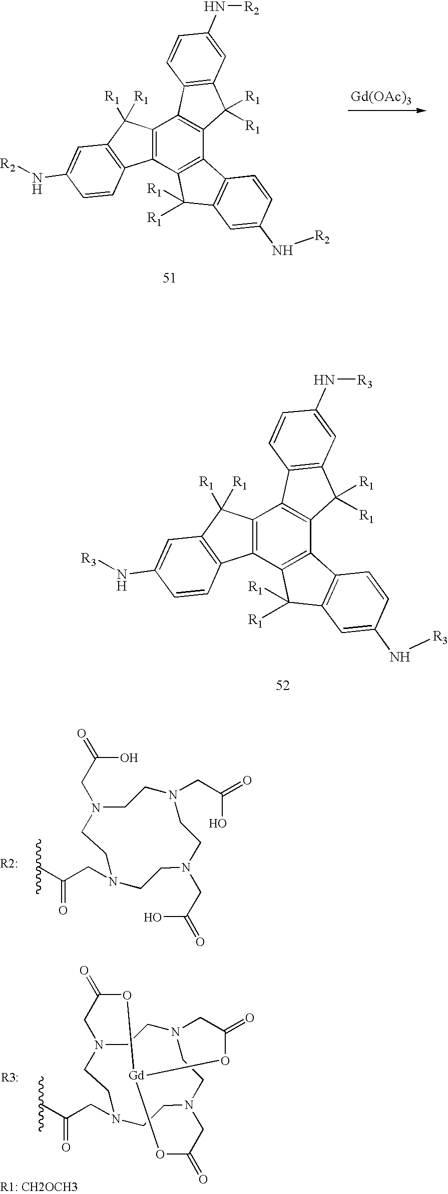

Compound 51 (26.4 g, 14.6 mmol) is dissolved in water and Gd(OAc)₃ (16.1 g, 48.2 mmol) is added. After 24 h the reaction mixture is concentrated and subjected to preparative HPLC purification to give compound 52.

What is claimed is:

1. Compound of formula (II) consisting of a core and groups —R-L-X′ attached to said core
A-(R-L-X′)n (II)
wherein
A denotes a rigid core;
R is the same or different and denotes a moiety that that constitutes an obstacle for the rotation of the covalent bond between the core A and R and/or the covalent bond between R and L and/or L and X, if L is present and/or the covalent bond between R and X′, if L is not present;
L is present or not and if present is the same or different and denotes a linker moiety;
X′ is the same or different and denotes a paramagnetic chelate consisting of a chelator X and a paramagnetic metal ion M; and
n denotes an integer of 3 or 4.

2. (canceled)

3. Compound according to claim 1 wherein A is an aromatic ring comprising at least 3 carbon atoms and optionally one or more heteroatoms N, S or O, said ring being optionally substituted with one or more of the following substituents: C₁-C₃-alkyl, optionally substituted with hydroxyl or amino groups, amino or hydroxyl groups or halogen, provided that there are n attachment points left for groups R-L-X.

4. Compound according to claim 1 wherein R is a slowly rotating moiety with a conformational lifetime of more than 0.1 μs.

5. Compound according to claim 1 wherein R is a moiety whose rotation is hindered by sterical interaction with the core A, and/or L, if present and/or X and/or other R groups.

6. Compound according to claim 1 wherein L is present.

7. Compound according to claim 1 wherein X is selected from residues of DOTA, DTPA, BOPTA, DO3A, HPDO3A, MCTA, DOTMA, DTPA BMA, M4DOTA, M4DO3A, PCTA, TETA, TRITA, HETA, DPDP, EDTA or EDTP.

8. Compound according to claim 1 wherein M is a paramagnetic ion of a transition metal or a lanthanide metal.

9. Compound according to claim 1 wherein all R are the same and/or all L, if present, are the same and/or all X′ are the same.

10. Composition comprising the compound according to claim 1 and at least one physiologically tolerable carrier.

11. Composition according to claim 10 for use as MR imaging agent or MR spectroscopy agent.

12. Use of the composition according to claim 10 as MR imaging contrast agent or MR spectroscopy agent.

13. Method of MR imaging and/or MR spectroscopy wherein the composition according to claim 10 is administered to a subject and the subject is subjected to an MR procedure wherein MR signals are detected from the subject or parts of the subject into which the composition distributes and optionally MR images and/or MR spectra are generated from the detected signals.

14. Method of MR imaging and/or MR spectroscopy wherein a subject which had been previously administered with the composition according to claim 10 is subjected to an MR procedure wherein MR signals are detected from the subject or parts of the subject into which the composition distributes and optionally MR images and/or MR spectra are generated from the detected signals.

15. Compounds of formula (I) consisting of a core and groups —R-L-X attached to said core
A-(R-L-X)n (I)
wherein
A denotes a rigid core;
R is present or not and if present is the same or different and denotes a moiety that that constitutes an obstacle for the rotation of the covalent bond between the core A and R and/or the covalent bond between R and L and/or L and X, if L is present and/or the covalent bond between R and X, if L is not present;
L is present or not and if present is the same or different and denotes a linker moiety;
X is the same or different and denotes a chelator; and
n denotes an integer of 3 or 4.

16. Method for the preparation of compounds according to claim 15 comprising
a) using as a first building block a core A that is substituted with reactive groups which allow for the attachment of R;
b) reacting R or a precursor thereof with said first building block to form a second building block consisting of the core A and R;
c) optionally reacting L or a precursor thereof with said second building block to form a third building block consisting of the core A, R and L; and
d) reacting X or a precursor thereof with said second or third building block.

17. Method for the preparation of compounds according to claim 15 comprising
a) using a first building block consisting of a core A and R fused or attached to A, wherein R is substituted with reactive groups which allow for the attachment of L or X;
b) optionally reacting L or a precursor thereof with said first building block to form a second building block consisting of the core A, R and L, and
c) reacting X or a precursor thereof with said first or second building block.

18. Method for the preparation of compounds according to claim 15 comprising
a) trimerisation or tetramerisation of a monomer comprising a moiety, which, upon trimerisation or tetramerisation forms A, said monomer further comprises R comprising a reactive moiety or a precursor thereof which allows for the attachment of L or a precursor thereof, if L is present, or X or a precursor thereof to form an intermediate;
b) optionally reacting L or a precursor thereof with said intermediate; and
c) reacting X or a precursor thereof with the intermediate or the reaction product of step b).

19. Method for the preparation of compounds according to claim 1 comprising carrying out any of the methods 16 to 18 and carrying out a subsequent step which comprises the complex formation of the reaction product of said any of the methods 16 to 18 with a suitable paramagnetic metal ion M
